(19) United States
(12) Patent Application Publication
(10) Pub. No.: US 2021/0311055 A1
(43) Pub. Date: Oct. 7, 2021

(54) System and Method for Disease Surveillance and Disease Severity Monitoring for COVID-19

(71) Applicant: NEW YORK UNIVERSITY, New York, NY (US)

(72) Inventors: John T. McDevitt, New York, NY (US); Michael P. McRae, Houston, TX (US); Nick Christodoulides, Austin, TX (US); Kritika Srinivasan, New York, NY (US)

(21) Appl. No.: 17/212,360

(22) Filed: Mar. 25, 2021

Related U.S. Application Data
(60) Provisional application No. 63/128,531, filed on Dec. 21, 2020.
(60) Provisional application No. 62/994,741, filed on Mar. 25, 2020.

(51) Int. Cl. G01N 33/569; G01N 33/68

(52) U.S. Cl. CPC G01N 33/56983; G01N 33/6854; G01N 2800/26; G01N 2333/75; G01N 2333/585; G01N 2333/58; G01N 2333/9123

(57) ABSTRACT

This disclosure describes portable bio-nano-chip assays, methods and compositions for diagnosing and assessing pathogen-mediated diseases or infections at point-of-care using biological samples. The assays, methods and compositions provide in a more convenient, less expensive, and less time-consuming sampling and analysis.

### CROSS REFERENCE TO RELATED APPLICATIONS

This application claims priority to U.S. Provisional Application No. 63/128,531, filed Dec. 21, 2020, and U.S. Provisional Application No. 62/994,741, filed Mar. 25, 2020, the contents of each of which are hereby incorporated by reference herein in their entirety.

### BACKGROUND OF THE INVENTION

The 2019-20 coronavirus pandemic is an ongoing global pandemic of coronavirus disease 2019 (COVID-19) caused by the severe acute respiratory syndrome coronavirus 2 (SARS-CoV-2). The virus was first reported in Wuhan, Hubei, China, on December 2019. On Mar. 11, 2020, the World Health Organization (WHO) declared the outbreak a pandemic. Based on WHO website's daily report on the outbreak, as of Mar. 19, 2020, over 209,839 cases have been confirmed in more than 168 countries and territories, with major outbreaks in mainland China, Italy, South Korea, and Iran. To date globally more than 10,000 people have died from the disease (World Health Organization). As of Jun. 15, 2020, about 8 million cases have been confirmed with approximately 435,000 deaths from the disease globally (Coronavirus Disease 2019 (COVID-19) Situation Report-133. World Health Organization, 1 Jun. 2020. Report No.: 133). However, there is expected to be a substantial under-reporting of cases, particularly of asymptomatic cases and in persons with milder symptoms. The COVID-19 crisis has exposed critical gaps in diagnostic testing and population-level surveillance (Sharfstein J M et al., 2020, JAMA 323(15):1437-8). With hospitalization rates of 20-31% and intensive care unit (ICU) admission rates of 5-12% (Morbidity and Mortality Weekly Report (MMWR), Severe Outcomes Among Patients with Coronavirus Disease 2019 (COVID-19)—United States, February 12-Mar. 16, 2020. [April 2020]), surges of patients requiring care have overwhelmed local healthcare systems and depleted reserves of medical resources. In Italy hospitals are so overwhelmed that ventilators are being rationed. This situation places physicians in extremely difficult situations relative to making life and death decisions.

Physicians are tasked with evaluating large amounts of rapidly changing patient data and making critical decisions in a short amount of time. Well-designed clinical decision support systems (CDSS) deliver pertinent knowledge and individualized patient information to healthcare providers to enhance medical decisions (The Office of the National Coordinator for Health Information Technology. Clinical Decision Support). Such systems may rely on surveys of similar cases, while others may use a “black box” approach (Wasylewicz ATM et al., 2019, Fundamentals of Clinical Data Science. Cham (CH): Springer; 2019. p. 153-69). Traditional scores like SOFA (Zhou F et al., 2020, Lancet 395(10229):1054-62; Seymour C W et al., 2016, JAMA 315(8):762-74; Vincent J L et al., 1996, Intens. Care Med. P. 707-10) and APACHE-2 (Zou, X et al., 2020, Crit. Care Med. 48(8):e657; Knaus, W A et al., 1985, Crit. Care Med. (10):818-29) are commonly used in hospitals for determining disease severity and mortality, whereas clinical decision management systems like electronic ICU (eICU) allow for systematic collection of comprehensive data (Lilly C M et al., 2014, CHEST. 145(3):500-7). However, CDSS that use conventional variables, such as demographics, symptoms, and medical history, often do not reach full diagnostic potential (Pollard, T J et al., 2018, Sci. Data. 5(1):180178).

Further, the economic impact of the coronavirus is mounting—with the Organization for Economic Co-operation and Development (OECD) warning the virus presents the largest danger to the global economy since the 2008 financial crisis (OECD Economic Outlook, Volume 2019, Supplement 2 ISSN: 16097408 (online)). For example, for the airline industry alone, according to the International Air Transport Association (IATA), it is predicted the COVID-19 outbreak will cost airlines $113 billion in lost revenue as fewer people take flights (www. weforum.org/agenda/2020/02/coronavirus-economic-effects-global-economy-trade-travel/). The economic impacts of quarantines and travel restrictions are probably more severe than the direct influence of death and illness.

The WHO has published several RNA-testing protocols for SARS-CoV-2 with the first issued in January 2020. The current gold standard method for COVID-19 disease diagnosis is based on RT-PCR with tests that can be done on either respiratory or blood samples. Results are generally available within a few hours to days, or, in some cases, results are communicated more than a week later. While access to reliable RT-PCR kits to date in the US has been problematic, aside from this gap in the supply chain the anticipated major stumbling block moving forward falls in the area of patient triage with the goal of identification of those few patients with high mortality probabilities.

Immunochromatographic strip (ICS) tests are commonly used for screening infectious diseases at the point-of-care. However, many ICS tests require manual readout of the test lines resulting in ambiguous test results with poor diagnostic sensitivity. While some ICS tests can improve sensitivity by using an automated instrument, these instruments are most often colorimetric and do not take advantage of the high signals and low backgrounds afforded to fluorescence immunoassays. Further, most instrumented ICS tests have reagents deposited over large spatial regions, or test lines, on a 1-dimensional substrate, resulting in inefficient capture with limited ability to detect low concentrations of antigen.

Further to assess disease severity and to help prioritize care for patients at elevated risk of mortality and manage low risk patients in outpatient settings or at home through self-quarantine, several scoring systems for COVID-19 severity have been developed or adapted from existing tools, such as the Brescia-COVID Respiratory Severity Scale (Duca A et al., 2020, Emerg. Med. Pract. 22(5 Suppl): CD1-CD2), African Federation for Emergency Medicine COVID-19 Severity Scoring Tool (Wallis, L A et al., 2020, Afr. J. Emerg. Med. 10(2):49), Berlin Criteria for Acute Respiratory Distress Syndrome (Rubenfeld, G D et al., 2012, JAMA 307(23):2526-33; Fan E. et al., 2018, JAMA 319(7):698-710), and Epic Deterioration Index (Singh, K et al., 2020, medRxiv. 1-22). However, these tools have either (a) not yet been externally validated in peer-reviewed publications or (b) developed specifically for COVID-19 patient populations.

There is thus a need in the art for compositions and methods for surveillance and severity score and monitoring of COVID-19 and patient mortality risk. The present invention addresses this unmet need in the art.

### SUMMARY OF THE INVENTION

In one aspect, the present invention provides a device comprising one or more bioaffinity ligands specific for one or more biomarkers of a pathogen-mediated infection or disease or the disease severity of the pathogen-mediated infection or disease. In one embodiment, In one embodiment, the pathogen-mediated infection or disease is COVID-19. In one embodiment, the device comprises an array of bead sensors, wherein each said bead sensor is a porous polymeric bead having an antibody or related bioaffinity ligand bound thereto. In one embodiment, the biomarker of COVID-19 is selected from the group consisting of IgG, IgM, and SARS CoV-2 spike. In one embodiment, the biomarker of COVID-19 disease severity is selected from the group consisting of: CRP, PCT, CK-MB, c-TN-I, D-dimer, and NT-proBNP. In one embodiment, the device further comprising internal microfluidics on said substrate for carrying fluid to and from said bead sensors. In one embodiment, the device further comprising a sample entry port. In one embodiment, the device further comprising at least one reagent blister fluidly connected to said bead sensors. In one embodiment, the device further comprising at least one waste fluid chamber fluidly connected to and downstream of said bead sensors. In one embodiment, the device further comprising positive and negative control bead sensors and calibrator bead sensors. In one embodiment, every said bead sensor is present in said array in at least duplicate. In one embodiment, every said bead sensor is present in said array in at least triplicate. In one embodiment, said antibody or bioaffinity ligand is conjugated to said bead sensor via a linker. In one embodiment, the device further comprising: a) one or more reagent chambers fluidly connected to and upstream of said array; and b) one or more waste fluid chambers fluidly connected to and downstream of said array; c) a sample inlet upstream and fluidly connected to said one or more reagent chambers; and d) wherein each bead sensor is a porous polymeric bead of size between 50-300 μm±10%.

In one aspect, the present invention provides an assay for diagnosing and assessing a pathogen-mediated disease or infection in a subject comprising: obtaining a biological sample from a subject; immunologically testing said sample to determine the level of one or more biomarkers of the pathogen-mediated infection or one or more biomarkers of the disease severity of the pathogen-mediated infection. In one embodiment, the pathogen-mediated infection or disease is COVID-19. In one embodiment, said testing is conducted on an array of agarose beads, conjugated to antibodies, and wherein signal from said array of agarose beads is analyzed by circular area of interest or line profiling or both. In one embodiment, the antibodies are specific for one or more biomarkers selected from the group consisting of: IgG, IgM, and SARS CoV-2 spike. In one embodiment, the antibodies are specific for one or more biomarkers selected from the group consisting of: CRP, PCT, CK-MB, c-TN-I, D-dimer, and NT-proBNP.

In one aspect, the present invention provides a diagnostic system comprising: a microfluidic lab-on-chip based immunoassay that comprises a disposable cartridge and a separate reader, wherein said cartridge fits into a slot on said reader, and said reader performs said immunoassay and outputs a result; said cartridge comprising: a generally flat substrate having embedded microfluidic channels connecting an inlet port to an embedded downstream assay chamber having a transparent cover and containing a removable array of bead sensors; ii) one or more reagent chambers fluidly connected to and upstream of said assay chamber; and iii) one or more waste fluid chambers fluidly connected to and downstream of said assay chamber; iv) wherein each bead sensor is a porous polymeric bead of size between 50-300 microns±10% having an antibody or bioaffinity ligand conjugated thereto, wherein said antibody or bioaffinity ligand is specific for a biomarker of a pathogen-mediated infection or the disease severity of a pathogen-mediated infection. In one embodiment, said antibody or bioaffinity ligand is specific for a biomarker selected from the group consisting of: IgG, IgM, SARS CoV-2 spike, CRP, PCT, CK-MB, c-TN-I, D-dimer, and NT-proBNP.

In one aspect, the present invention provides a kit comprising a cartridge wrapped in an airtight package.

In one aspect, the present invention provides a method for diagnosing or treating a pathogen-mediated disease or infection, the method comprising obtaining a biological sample from a patient; and immunologically testing said sample to determine the of level of one or more biomarkers of the pathogen-mediated infection or one or more biomarkers of the disease severity of the pathogen-mediated infection. In one embodiment, the pathogen-mediated infection or disease is COVID-19. In one embodiment, said testing is conducted on an array of agarose beads. In one embodiment, the biomarker of COVID-19 is selected from the group consisting of IgG, IgM, and SARS CoV-2 spike. In one embodiment, the biomarker of COVID-19 disease severity is selected from the group consisting of: CRP, PCT, CK-MB, c-TN-I, D-dimer, and NT-proBNP. In one embodiment, the method further comprises assigning a risk-stratification to the patient. In one embodiment, the method further comprises performing an optimal clinical intervention, when the level of the one or more biomarkers are above a threshold level.

In one aspect, the present invention provides a method for screening a subject for the probability of SARS-CoV2 infection, comprising calculating a screening score for the subject, wherein the screening score is based upon a logistic regression model of one or more environmental, physiological, or demographic factors of the subject. In one embodiment, the subject is a patient scheduled for a dental or medical procedure. In one embodiment, the one or more environmental, physiological, or demographic factors of the subject comprises one or more of: body temperature, SpO2, race/ethnicity, local positivity rate of the subject's residence, case incidence rate of the subject's residence. In one embodiment, the logistic regression model is a lasso logistic regression model. In one embodiment, the method further comprising obtaining a sample of the subject when the score surpasses a threshold; and assaying the sample for one or more antigens associated with SARS-CoV2 infection and one or more antibodies associated with SARS-CoV2 infection. In one embodiment, assaying comprises contacting the sample to a point-of-care device that sequentially assays for the one or more antigens and the one or more antibodies.

In one aspect, the present invention provides a system for detecting SARS-CoV2 infection in a subject, the system comprising a point-of-care device that detects one or more antigens associated with SARS-CoV2 infection and one or more antibodies associated with SARS-CoV2 infection. In one embodiment, the device is configured to sequentially assay for the one or more antigens and the one or more antibodies.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 depicts a schematic of the intended use cycle of the programmable bio-nano-chip (p-BNC) system. This is a flexible platform for digitizing biology, featuring sensor ensembles that measure biomarkers in highly efficient manner.

FIG. 2 depicts an exemplary schematic of a cartridge comprising a plurality of agarose beads at discrete locations, where each bead comprises an affinity ligand specific for a biomarker of COVID-19 or COVID-19 disease severity.

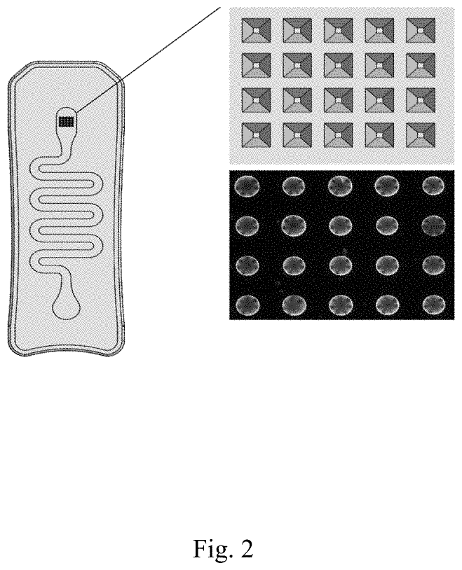

FIG. 3 depicts clinical decision support system and mobile app for managing COVID-19 care.

FIG. 4A through FIG. 4B depict Tier 1 Outpatient Model results. Lasso logistic regression coefficients. FIG. 4A reveals the relative predictor importance in generating the score. FIG. 4B depicts the box/scatter plot from internal validation that shows Tier 1 Outpatient Scores for the four outcomes. A cutoff score of 18 (red dotted line) balances sensitivity and specificity for “Non-case” vs. “Case” patients (gray line) (No Hosp.=patients who were not hospitalized, Vent.=patients who were ventilated, CV comorbidities=cardiovascular comorbid conditions).

FIG. 5A through FIG. 5B depict Tier 2 Biomarker Model results. FIG. 5A depicts Lasso logistic regression coefficients that reveals relative predictor importance in generating the score. FIG. 5B depicts the box/scatter plot from internal validation that shows Tier 2 Biomarker Scores for the three patient outcomes. A cutoff score of 27 (horizontal red dotted line) balances sensitivity and specificity for “Non-case” vs. “Case” patients (vertical gray line) (No Hosp.=patients who were not hospitalized).

FIG. 6A through FIG. 6B depict external validation results. FIG. 6A depicts the Tier 1 Outpatient Model that was evaluated on data from COVID-19 patients at Zhongnan Hospital of Wuhan University (Guo, T et al., 2020, JAMA Cardiol.). FIG. 6B depicts the Tier 2 Biomarker Model that was evaluated on data from COVID-19 patients at Tongji Hospital (Yan L. et al., 2020. Nat. Mach. Intell. 2(5):283-8).

FIG. 7 depicts spaghetti plot of longitudinal COVID-19 Biomarker scores for patients in the external validation set from Tongji Hospital (Yan L. et al., 2020. Nat. Mach. Intell. 2(5):283-8) between January 10 and Feb. 18, 2020. These data represent individual patients' scores over a median interquartile range (IQR) of 12.5 (8-17.5) days between admission and outcomes of discharged or deceased. The first scores available after admission were significantly higher in those that died vs. those that were discharged (AUC 0.97, cutoff score of 19), and over time patients who were discharged had an average decrease in score (−4.7) while those that died had an average increase in score (+11.2).

FIG. 8 depicts clinical decision support system for COVID-19 screening. Prior to entering the dental office, patients may be screened for the presence of one or more symptoms (fever, cough, and shortness of breath) of COVID-19. If symptomatic, patients should be requested to reschedule their appointments for a later date. The Pre-screening Algorithm (Tier 0) helps determine if a patient is eligible for COVID-19 screening. Patients with a high pre-screening score are recommended for the POC antigen/antibody screening in the dental setting. Beyond the scope of this work and published elsewhere are prognostic models (Tier 1 and Tier 2) for predicting COVID-19 mortality in inpatient, outpatient, and hospital settings (McRae, M P et al., 2020, J. Med. Internet Res. 22(8):e22033).

FIG. 9A through FIG. 9D depicts model development results showing lasso logistic regression coefficients for the full model with local positivity rate, temperature, SpO2, race, and ethnicity (FIG. 9A), receiver operating characteristic (ROC) curve for the same model (FIG. 9B), univariate AUC values for predictors categorized by predictor type (environmental, physiological, race/ethnicity, and combination) (FIG. 9C), and box/scatter plot of the resulting scores from internal validation (FIG. 9D).

FIG. 10 depicts diagnostic models for discriminating COVID-19 positive vs. negative (RT-PCR) in asymptomatic/pre-symptomatic individuals. The CIR-only model is the preferred pre-screening model (red bar). Temp. is body temperature ≥99° F. SpO2 is oxygen saturation ≤96%. CIR is the case incidence rate. LPR is the local positivity rate.

FIG. 11A through FIG. 11E depicts the POC microfluidics-based combination antigen/antibody assay tool. Illustration of assay cartridge (FIG. 11A) shows an array of 20 programmable agarose bead sensors (FIG. 11B), with antigen and antibody capture beads imaged separately at steps 6 and 9 of assay (see FIG. 9 for sequence of fluidic steps), respectively, and stitched together to constitute the final image. The bead sensor serves as a high surface area substrate for developing programmable immunoassays for COVID-19 antigen and antibody detection (FIG. 11C). Multiplexed fluorescent images show bead sensor arrangement and captured analyte via fluorescence, with variation in signal intensity at various concentrations (FIG. 11D). Averaged bead fluorescence intensity (MFI) from the multiplexed assays were used to calibrate standard curves for the antigen and antibody tests (FIG. 11E).

FIG. 12 depicts an exemplary patient data flow.

FIG. 13A through FIG. 13B depict test positivity rates (FIG. 13A) and case incidence rates (FIG. 13B) from New York State Department of Health for the three counties in which the NYU Family Health Centers are located. While the figures below show daily changes in positivity and incidence, the models developed in this study used 7-day averaged rates prior to the patient's encounter (ie, averaged 1-8 days before encounter).

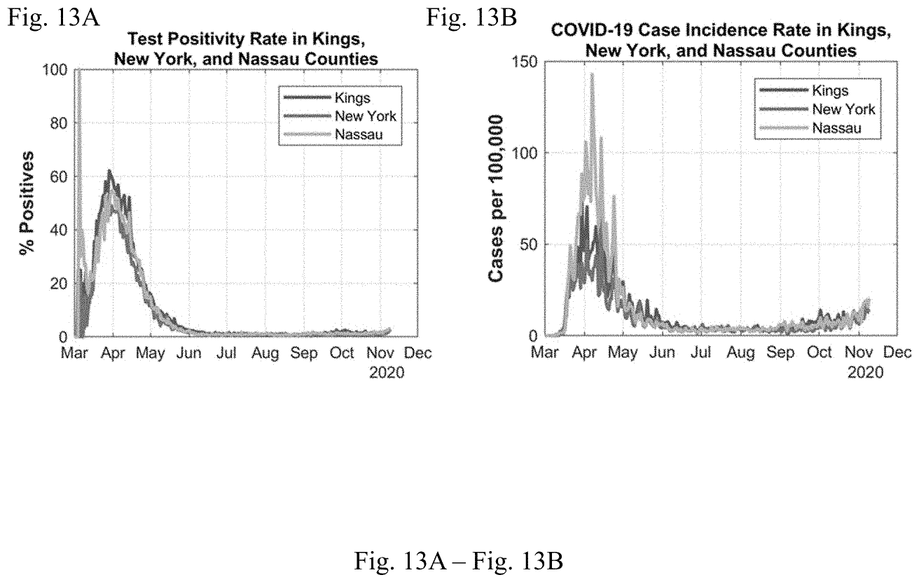

FIG. 14A through FIG. 14F depict cartridge and instrument evolution shown for the following stages: FIG. 14A depicts non form factor flow cell serviced with syringe pumps and imaged by commercial epi-fluorescence microscope, FIG. 14B depicts non form factor laminate prototype serviced with syringe pumps and imaged by commercial epi-fluorescence microscope, FIG. 14C depicts form factor laminate prototype serviced with syringe pumps and imaged by commercial epi-fluorescence microscope, FIG. 14D depicts form factor laminate prototype with embedded blister packs and imaged by commercial epi-fluorescence microscope, FIG. 14E depicts form factor laminate prototype with embedded blister packs and imaged by monorail customized epi-fluorescence image station, FIG. 14F depicts production ready cartridge and analyzer instrumentation suitable for point of care measurements. The use of multiple stages of image instrumentation and cartridge has allowed for the various subsystems to be tested and key subcomponents to be isolated. At the time of this submission fully integrated instrumentation shown in FIG. 14F is available for drug testing applications. This instrumentation is designed to be programmable allowing for its adaptation to other applications including COVID-19 duplex testing.

FIG. 15 depicts clinical decision support system for COVID-19 diagnosis and prognosis across a spectrum of the disease and for multiple care settings. In scope of this work is the Pre-screening Algorithm (Tier 0) to determine if a patient is eligible for COVID-19 screening and the POC antigen/antibody screening in the dental setting. Beyond the scope of this work and published elsewhere are prognostic models (Tier 1 and Tier 2) for predicting COVID-19 mortality in inpatient, outpatient, and hospital settings (McRae, M P et al., 2020, J. Med. Internet Res. 22(8):e22033).

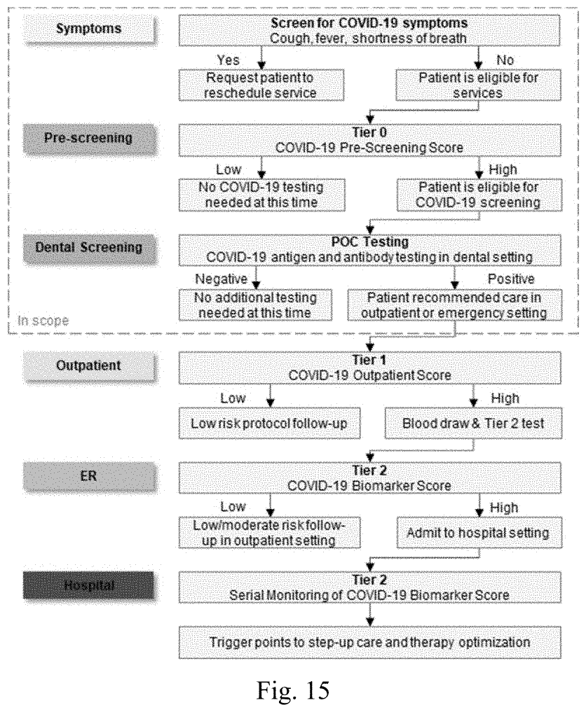

FIG. 16 depicts COVID-19 antigen/antibody assay sequence. Step 1 shows the sample (antigen+/−antibody) loaded to the cartridge input port, followed by sample delivery over the bead array through buffer flow via right blister (Step 2) and finished with a wash step (Step 3). Step 4 shows introduction of the antigen detection reagent conjugated to Alexa Fluor 488 (Step 4B) via the right reagent pad, over the bead array, followed by incubation (Step 5) and wash (Step 6) steps. In the presence of SARS-CoV-2 nucleocapsid antigen in the sample, the post-assay completion image shows antigen capture beads fluorescing as a result of the antigen immune-complex formation (Step 6B). Finally, Step 7 shows the introduction of the antibody detection reagent conjugated to Alexa Fluor 488 (Step 7C) via the left reagent pad over the bead array, followed by final incubation (Step 8) and final wash (Step 9) steps. In the presence of SARS-CoV-2 IgG1 antibody in the sample, the post-assay completion image shows the antibody capture beads fluorescing as a result of the antibody immune-complex formation (Step 9C).

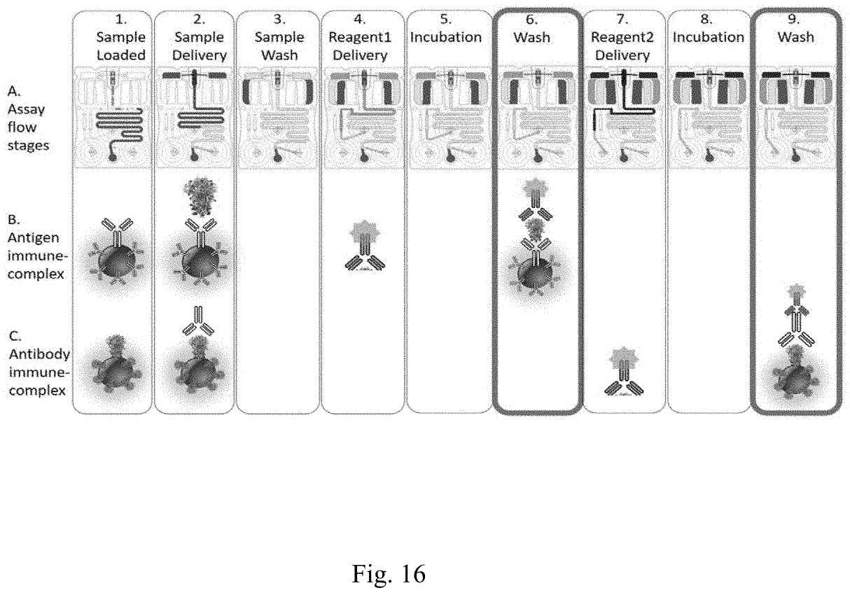

### DETAILED DESCRIPTION

The invention generally relates to devices, systems, and methods for detecting of a pathogen, diagnosing a pathogen-mediated infection or disease, assessing the risk of having a pathogen-mediated infection or disease, and assessing the disease severity of a pathogen-mediated infection or disease. For example, in certain aspects, the present invention relates to the detection of a respiratory pathogen and associated disease, and assessing the disease severity of a respiratory pathogen-mediated infection or disease. In one embodiment, the invention relates to detection of a respiratory pathogen and associated disease, including asymptomatic and subclinical infections. In one embodiment, the invention relates to the detection of pathogens, including existing pathogens and novel pathogens, that can cause acute respiratory distress syndrome (ARDS). While the following description may focus upon the detection of SARS CoV-2 and COVID-19 and assessing COVID-19 disease risk or severity, the present invention encompasses detection of other pathogens that may lead to respiratory conditions, such as ARDS. For example, the invention also relates to the diagnosis and disease severity assessment of influenza infection, SARS, MERS, RSV infection, enterovirus infection, rhinovirus infection, adenovirus infection, parainfluenza infection, and any other viral, bacterial, or pathogenic disease or infection that can cause severe respiratory conditions or ARDS.

In one aspect, the present invention provides point of care diagnostics for pathogens and pathogen-mediated infection or disease, devices containing biomarker specific reagents, portable devices for use as analyzers or drivers with same, software to evaluate and report test results, and the overall diagnostics and reporting system as a whole.

Signs of pneumonia may precede confirmation of COVID-19 infection through RT-PCR. Early detection of exposed or infected individuals, especially those that are asymptomatic, and disease severity are both important so as (1) to prevent transmission to others, thereby mitigating the effects of this pandemic, and (2) to enable prompt implementation of appropriate treatments, so that ultimately lives may be saved. Here, a point-of-need solution that provides near real-time results is needed. Thus, while there are tools for disease diagnosis based on RT-PCR, there remains a huge gap in determining disease prognosis, especially with respect to early identification of key individuals that are at elevated risk of mortality. Access of such tools for use at the point of care and for use in low- and middle-income countries would help to manage this disease on a global basis.

As described herein, a portable assay platform for COVID-19 diagnostics fulfills significant testing gaps today in clinical settings (hospitals, clinics, and laboratories) and deployed public settings at risk for community spread, such as businesses, schools, airports, and train stations. In one aspect, the invention provides a programmable bio-nano-chip (p-BNC)-based assay for detecting the presence, level, or concentration of one or more particular biomarkers in a biological sample. In certain aspects, the one or biomarkers are indicative of the presence of SARS-CoV-2, COVID-19, or one or more underlying medical conditions that contribute to the severity of COVID-19. Such a chip can be used with the laboratory-based p-BNC instrumentation, the portable p-BNC assay system or a hand-held device designed for point-of care use.

The p-BNC is a packaged microfluidic sample processing and immune-analysis chip that serves as the functional component for the detection and quantitation of the one or more biomarkers.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

By “reader” or “detector” or “analyzer” what is meant is a device that contains the optics, optic sensing means, processor, user interface, and fluidics and is the device that runs the assays described herein and thus “analyzes” the sample and “reads” or “detects” the results.

By “card” or “cartridge” what is meant is a generally planar substrate having microfluidic channels and chambers therein, as well as one or more access ports, and houses the bead array specific for the assays described herein.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

It is understood that in certain embodiments and examples, an antibody as described may be replaced with any bioaffinity ligand. Suitable bioaffinity ligands include any molecule that binds to a biomarker of interest. Exemplary bioaffinity ligands include, but are not limited to, antibodies, antibody fragments, proteins, peptides, peptidomimetics, nucleic acid molecules, bacteriophages, aptamers, and small molecules.

By the term “specifically binds,” as used herein with respect to an antibody or bioaffinity ligand, is meant an antibody or bioaffinity ligand which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the term “marker” or “biomarker” is meant to include a parameter which is useful according to this invention for determining the risk, presence and/or severity of COVID-19.

The term “control or reference standard” describes a material comprising none, or a normal, low, or high level of one of more of the marker (or biomarker) expression products of one or more the markers (or biomarkers) of the invention, such that the control or reference standard may serve as a comparator against which a sample can be compared.

As used herein, an “immunoassay” refers to a biochemical test that measures the presence or concentration of a substance in a sample, such as a biological sample, using the reaction of an antibody to its cognate antigen, for example the specific binding of an antibody to a protein. Both the presence of the antigen or the amount of the antigen present can be measured.

The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The “level” of one or more biomarkers means the absolute or relative amount or concentration of the biomarker in the sample.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Sample” or “biological sample” as used herein means a biological material isolated from an individual, including but is not limited to organ, tissue, exosome, breast milk, blood, plasma, saliva, urine and other body fluid. The biological sample may contain any biological material suitable for detecting the desired biomarkers, and may comprise cellular and/or non-cellular material obtained from the individual.

As used herein, an “instructional material” includes a publication, a recording, a diagram, or any other medium of expression which can be used to communicate the usefulness of a device, system, or method of the present invention. The instructional material of the kit of the invention can, for example, be affixed to a container which contains the device or system of the invention or be shipped together with a container which contains the device or system. Alternatively, the instructional material can be shipped separately from the container with the intention that the instructional material and the device or system be used cooperatively by the recipient.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is related to devices, systems, and methods for diagnosing and assessing disease risk or severity of pathogen-mediated diseases and infection, including, but not limited to COVID-19, SARS, MERS, influenza, and the like. The present invention can be used to detect the presence of a pathogen-mediated infection or disease in a subject, assess the risk of having a pathogen-mediated infection or disease, and assess the disease severity of the pathogen-mediated disease of the subject. The present invention can be used to detect the presence of subclinical infections in a subject and reduce exposure risk to medical community.

While the present description may focus on aspects related to COVID-19, it should be understood that the present invention relates to any pathogen-mediated disease or infection, particularly those that may lead to severe respiratory conditions such as ARDS. For example, the devices and tools described herein can be quickly adapted and repurposed to manage infections from other novel or existing pathogens, such as those respiratory pathogens that can cause ARDS.

Symptoms of COVID-19 are non-specific and those infected may either be asymptomatic or develop flu-like symptoms such as fever, cough, fatigue, shortness of breath, or muscle pain. Further development can lead to severe pneumonia, acute respiratory distress syndrome, sepsis, septic shock, and death. (www. cdc.gov/coronavirus/2019-ncov/symptoms-testing/symptoms.html?CDC.html; WHO-China Joint Mission (16-24 Feb. 2020). “Report of the WHO-China Joint Mission on Coronavirus Disease 2019 (COVID-19)” (PDF). World Health Organization. Retrieved 14 Mar. 2020) Some of those infected may be asymptomatic, returning test results that confirm infection, but show no obvious clinical symptoms. Those with close contact to confirmed infected people should be closely monitored and examined to rule out infection. The usual incubation period (the time between infection and symptom onset) ranges from one to fourteen days, but most commonly it is around five days (WHO-China Joint Mission (16-24 Feb. 2020). “Report of the WHO-China Joint Mission on Coronavirus Disease 2019 (COVID-19)” (PDF). World Health Organization. Retrieved 14 Mar. 2020). Prompt identification of COVID-19 exposure/infection is critical to slowing spread of the disease.

The present invention relates to a panel of biomarkers for disease surveillance of COVID-19 and disease severity monitoring of COVID-19. In one aspect, the invention comprises systems and methods for detection of a first panel of biomarkers to assess the presence of SARS CoV-2 and/or COVID-19 in a subject. In one embodiment, the first panel of biomarkers comprises one or more biomarkers indicative of an immune response. For example, in one embodiment, the first panel of biomarkers comprises IgM, which is indicative of active disease and is produced immediately after exposure to a particular antigen. In one embodiment, the first panel of biomarkers comprises IgG, which is indicative of past disease and represents the late stage response. In one embodiment, the first panel of biomarkers comprises one or biomarkers of the pathogen, such as a protein, nucleic acid molecule or antigen of the pathogen. For example, in the context of SARS CoV-2, the first panel of biomarkers comprises a biomarker of SARS CoV-2, including any viral protein or viral nucleic acid, such as SARS CoV-2 spike protein (e.g., spike antigen), the S1 or S2 subunits of the SARS CoV-2 spike protein, or the SARS CoV-2 nucleocapsid protein (N-protein). In one embodiment, the first panel of biomarkers comprises 1, 2 or 3 of: IgM, IgG, and SARS CoV-2 spike.

In one aspect, the invention relates to systems and methods for detection of a second panel of biomarkers to assess disease severity. For example, in certain embodiments, the second panel of biomarkers comprises one or more biomarkers associated with mortality. In one embodiment, the second panel of biomarkers comprises C-reactive protein (CRP), which is an inflammatory marker and is an indicator of mortality. In certain aspects, the second panel of biomarkers comprises one or more biomarkers associated with underlying conditions, such as acute respiratory illness, cardiac failure, and renal dysfunction. In a recent study, clinical data on 82 death cases laboratory-confirmed as SARS-CoV-2 infection, respiratory failure remained the leading cause of death (69.5%), following by sepsis syndrome/MOF (28.0%), cardiac failure (14.6%), hemorrhage (6.1%), and renal failure (3.7%). Furthermore, respiratory, cardiac, hemorrhage, hepatic, and renal damage were found in 100%, 89%, 80.5%, 78.0%, and 31.7% of patients, respectively. [Bicheng Zhang, el. Clinical characteristics of 82 death cases with COVID-19. medRxiv 2020.02.26.20028191; doi: doi.org/10.1101/2020.02.26.20028191.] In one embedment, the second panel of biomarkers comprises one or more of: procalcitonin (PCT), Creatine Kinase myocardial b fraction (CK-MB), Cardiac troponin I (c-TN-I), D-dimer, and N-terminus pro B-type natriuretic peptide (NT-proBNP), each of which are markers of heart attacks and/or cardiac failure. In one embodiment, the second panel of biomarkers comprises 1, 2, 3, 4, 5 or 6 of CRP, PCT, CK-MB, c-TN-I, D-dimer, and NT-proBNP.

In one embodiment, the present invention provides systems and methods for detection of a first panel of biomarkers comprising 1, 2, or 3 of IgM, IgG, and SARS CoV-2 spike; and detection of a second panel of biomarkers comprising 1, 2, 3, 4, 5, or 6 of: CRP, PCT, CK-MB, c-TN-I, D-dimer, and NT-proBNP. In one embodiment, the present invention provides systems and methods for detection of a first panel of biomarkers comprising IgM, IgG, and SARS CoV-2 spike; and detection of a second panel of biomarkers comprising CRP, PCT, CK-MB, c-TN-I, D-dimer, and NT-proBNP.

In some embodiments, the analysis may be performed using a hand-held device with disposable chip that provides a rapid, cost effective, yet sensitive method of detecting these markers of COVID-19 and COVID-19 disease severity. Additionally, because of its portability, low cost, and speed, this approach can function in point of care settings using noninvasive samples, including, but not limited to brush biopsy samples, blood samples (whole blood, serum, and plasma samples), saliva samples, and urine samples. The invention therefore also includes the disposable chip with reagents placed thereon that are specific for measuring the above markers. In some embodiments, the device contains power, detection of signal, programming, and capacity to display the final results.

Described herein is a transformative diagnostic technology based on handheld assay platform for COVID-19 that addresses the above-mentioned significant gaps in testing technology. The analyzer here developed features: improved sensitivity and lower backgrounds through fluorescence detection; improved optical signal transmission via a high numerical aperture (NA) imaging system not feasible with typical ICS tests; intuitive user interfaces intended for nonexperts; and “walk-away mode” test results in as little as 3 minutes.

There are two overarching testing goals for this key effort. The first allows for population-based disease surveillance for community preparedness as measured through the simultaneous measurement of one or more of: IgG, IgM and SARS COV-2 spike. Immunoglobulin antibodies appear soon after infection and initiate immune response in the affected patient. IgG represents the late stage response to a disease whereas IgM is produced immediately after the exposure to a particular antigen.

The second panel involves development of a prognostic quantitative multiplexed diagnostic panel that can be used to predict disease severity for patients suffering from COVID 19 infections. This novel diagnostic capability is currently lacking in all commercial approaches and has the potential to have a transformative influence on the management of COVID-19 disease. This essential panel here designated will involve simultaneous measurement of one or more of: CRP, NT-proBNP, D-dimer, procalcitonin, CK-MB and c-Tn-I.

| Panel | Analytes | Comments |
|---|---|---|
| Exposure | COV 2 (spike antigen) | Direct evidence of exposure |
| | IgM (active disease) | Produced immediately after the exposure to a |
| | | particular antigen |
| | IgG (past disease) | Represents the late stage response to a disease |
| Severity | Biomarkers | |
| | CRP | inflammatory marker; indicator of mortality |
| | PCT | elevated in COVID 19 patients |
| | CK-MB | heart attacks, cardiac failure |
| | c-TnI | heart attacks, cardiac failure |
| | D-dimer | cardiac failure |
| | NT-proBNP | cardiac failure |
| | Age & Risk Factors | |
| | CVD | |
| | Diabetes | |
| | Lung disease | |
| | Age | |

A COVID-19 exposure chip can reliably detect the presence of SARS CoV-2 and/or COVID-19 in a subject, regardless of whether the subject is showing clinical symptoms. Thus, the chip can be used to determine whether the subject should be quarantined or isolated from the rest of the community. Further, the chip can be used to identify additional persons for testing, such as persons that may have come in contact with the subject or occupied a space in which the subject occupied. Thus, the exposure chip provides a reliable method for COVID-19 disease surveillance.

A COVID-19 disease severity chip can reliably identify a subject as having a likelihood to develop severe disease that could lead to morbidity or mortality. For example, the disease severity chip can be used to provide a prognosis or a risk for developing severe complications. In some embodiments, the disease severity chip can aid in identifying those subjects who will likely need close monitoring, hospitalization, intensive care, ventilators, or therapeutic agents. Thus, in some embodiments, the disease severity chip can aid in allocating scarce resources among large number of subjects who test positive for having COVID-19.

In some embodiments, the first panel of biomarkers and second panel of biomarkers are assessed using a single cartridge or chip, where affinity ligands specific for markers of both panels are present on the single chip. In some embodiments, the first panel of biomarkers and second panel of biomarkers are assessed using different cartridges or chips, where the first panel is assessed using a first cartridge or chip having affinity ligands specific for biomarkers of the first panel; and where the second panel is assed using a second cartridge or chip having affinity ligands specific for the biomarkers of the second panel.

Programable Bio-Nano-Chip

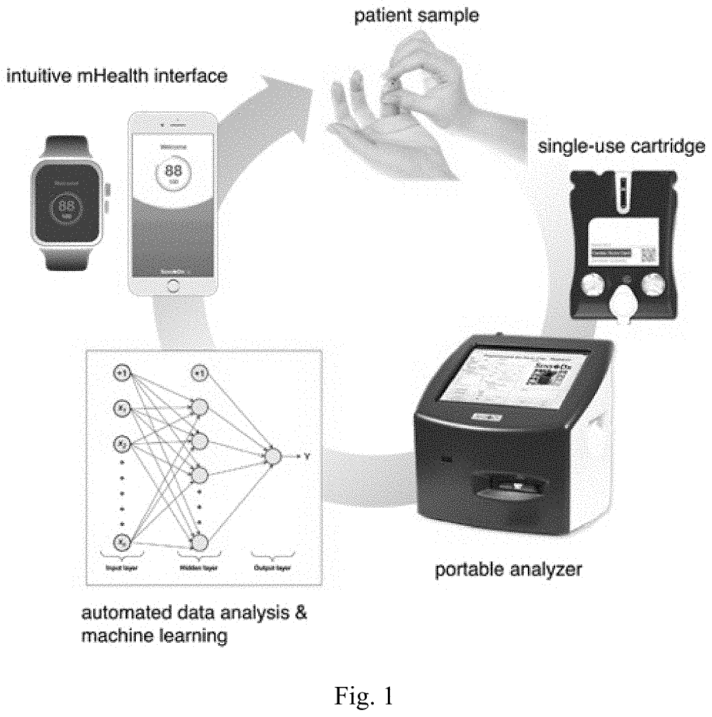

In one aspect, the invention provides a Programable Bio-Nano-Chip (p-BNC) that allows for the analysis of a biological fluid for the diagnosis and management of subjects having or at risk for having a pathogen-mediated disease or infection, such as COVID-19. The p-BNC system allows for the simultaneous quantification of expression of multiple molecular biomarkers of the pathogen-mediated disease or infection and/or disease severity of the pathogen-mediated disease or infection in an automated manner using refined image analysis algorithms based on pattern recognition techniques and advanced statistical methods (see e.g., FIG. 1). In certain embodiments, the device has at least 90% specificity and 90% sensitivity, preferably at least 92, 93, 94, 95, 96, or 97%.

In one embodiment, the invention provides a device comprising at least one bioaffinity ligand bound thereto, wherein said bioaffinity ligand is specific for a target selected from IgM, IgG, SARS CoV-2 spike, CRP, PCT, CK-MB, c-TN-I, D-dimer, and NT-proBNP. In one embodiment, the device comprises a plurality of types bioaffinity ligands, specific for the set of targets of IgM, IgG, SARS CoV-2 spike, CRP, PCT, CK-MB, c-TN-I, D-dimer, and NT-proBNP. Exemplary bioaffinity ligands include, but are not limited to, antibodies, antibody fragments, proteins, peptides, peptidomimetics, nucleic acid molecules, bacteriophages, aptamers, and small molecules.

In one embodiment, the invention provides a testing cartridge comprising a generally flat substrate having thereon individual bead sensors arranged in an array, wherein each bead sensor is a porous polymeric bead having at least one bioaffinity ligand bound thereto, wherein said bioaffinity ligand is specific for a target selected from IgM, IgG, SARS CoV-2 spike, CRP, PCT, CK-MB, c-TN-I, D-dimer, and NT-proBNP.

In one embodiment, the testing cartridge further comprises internal microfluidics on said substrate for carrying fluid to and from said bead sensors. In one embodiment, the testing cartridge further comprises a sample entry port. In one embodiment, the testing cartridge further comprises at least one reagent blister fluidly connected to said bead sensors. In one embodiment, the testing cartridge further comprises at least one waste fluid chamber fluidly connected to and downstream of said bead sensors. In one embodiment, the testing cartridge further comprises positive and negative control bead sensors and calibrator bead sensors having known amounts of a target antigen being calibrated.

In one embodiment, every bead sensor is present in said array in at least duplicate. In one embodiment, every bead sensor is present in said array in at least triplicate. In one embodiment, the antibody is conjugated to said bead sensor via a linker.

In one embodiment, the invention provides a testing cartridge further comprising one or more of the following: one or more reagent chambers fluidly connected to and upstream of said array; one or more waste fluid chambers fluidly connected to and downstream of said array; a sample inlet upstream and fluidly connected to said one or more reagent chambers; and wherein each bead sensor is a porous polymeric bead of size between 50-300 μm±10%.

In one embodiment, the diagnostic is performed on a portable device together with disposable biochips, that contains various liquid and/or dried reagents. The analyzer device contains microfluidics for sample and reagent flow, means for detecting signals, usually light-based signals, computing means for analyzing collected data and usually means for inputting patient information and displaying final results.

In one embodiment, the disposable lab cards or cartridges contain a detection window which has a membrane therein sized to capture cells. In one embodiment, the membrane is exchangeable, e.g., with membranes of differing size, or with arrays of antibodies, and thus is contained inside a hinged door or lid or similar components that serves to lock the exchangeable component into the card.

In certain embodiments, the cartridges can be used to analyze and image whole cells. In one embodiment, an inlet port is fluidly connected to the detection window, and sample is applied and travels to the window where cells are trapped by the membrane. In one embodiment, the cartridge further comprises regent chambers, and the reader activates the reagent chamber, pushing wash fluid to the assay chamber to wash away cell debris as needed. Next, a second reagent chamber is activated, and travels past a dry pad or chamber containing dry bioaffinity ligands (e.g. antibodies) and stains, reconstitutes same and carries these to the assay chamber, where the cells are stained with nuclear, cytoplasmic and antibody stains. Optionally, these reagents can be premixed with the second chamber fluid. In one embodiment, the stability of antibody components is improved in the dry form. In one embodiment, the dry pads are exchangeable, e.g. via a hinged lid. The excess reagents can then be washed away, using wash from the first chamber, and the remaining signals detected and analyzed. Additional assay chambers can be provided, depending on the number of analytes to be analyzed and the spectral range of the signals (and device capacity to distinguish same). Alternatively, the cells can be serially stained, and then washed clean and re-stained.

Compared to gold standard methods, such as enzyme-linked immunoassay (ELISA), the p-BNC system exhibits assay times in minutes instead of hours, limits of detection (LOD) two or more orders of magnitude lower, and a proven capacity to multiplex 5 or more concurrent analytes with appropriate internal controls and calibrators. For example, salivary biomarkers that were previously undetectable by standard methods, may now be targeted with the portable testing devices to assess systemic disease in a non-invasive fashion. Examples of such devices are set forth in Goodey et al., J. Amer. Chem. Soc., 123(11):2559-2570, 2001, and Christodoulides et al., Lab. Chip, 5(3):261-9, 2005b, the entire contents of which are incorporated by reference into this application.

The strong analytical performance of the p-BNC system may be attributed to the porous nature of its agarose bead sensors, the active transport mode of delivery of the sample and detection reagents, as well as the highly stringent washes associated with this micro-fluidic approach. Like ELISA, the bead-based p-BNCs complete two-site immunometric, as well as competitive, immunoassays; however, unlike ELISA, which limits the diffusion-mediated antigen (Ag)-Antibody (Ab) binding to a 2-dimensional, planar surface at the bottom of the well, the p-BNC cards provide a ˜1,000 to 10,000-fold increase in surface area on the 3-dimensional bead or disk sensor. This 3-dimensional reactor allows for significantly increased contact area, as well as on, off and then on again, higher avidity Ag-Ab interactions. All of the afore-mentioned features contribute to the generation of high signal-to-noise ratios, which ultimately translate into the advanced detection capabilities associated with the p-BNC system.

In one embodiment, the invention is directed to a disposable cartridge, cassette, or lab card, wherein the testing sites comprise agarose substrates (beads or disks) that are conjugated to either target or anti-target antibody, and thus serves in competitive or sandwich two-site immunometric assays. In one embodiment, the agarose substrates are agarose beads. In one embodiment, the agarose substrate is conjugated to an anti-target antibody. In one embodiment, the anti-target antibody is specific for a target selected from IgM, IgG, SARS CoV-2 spike, CRP, PCT, CK-MB, c-TN-I, D-dimer, and NT-proBNP.

The cartridge comprises channels and other microfluidics, such that fluid can be forced to pass through the agarose beads or disk. Blister packs or other chambers can also be placed on the cartridge and can contain, e.g., wash fluids, reagent fluids, and the like. Channels designed for mixing and fluid flow permeate this architecture, and manipulations of the fluidic cartridges reconstitute and disperse reagents through the lab card. Linear actuation controls all fluid motion via pressure actuation steps provided by the analyzer device.

In more detail, a sample entry port is fluidly connected via microfluidics to the assay chamber. In certain embodiments, the assay chamber comprises a plurality of bead sensors as described herein. In certain embodiments, the assay chamber is addressable from the exterior of the cartridge to allow for insertion of an array of bead sensors into the assay chamber; thereby allowing for different arrays of bead sensors (i.e. different arrays specific for different markers and indications) to be swapped in and out of the assay chamber. The assay chamber is either open to the environment or comprises a transparent lid to allow for imaging and image analysis of the cells within assay chamber. In certain embodiments, one or more pinch valves function to allow controlled delivery of microfluidic elements. In some embodiments, buffer entry ports are fluidly connected to microfluidics of the cartridge. In certain embodiments, one the cartridge comprises one or more blister packs that contain liquid reagents, such as wash buffers. Blister packs allow for a self-contained cartridge with a smaller footprint. Alternatively, the device could be connected directly to an external fluid source via buffer entry ports. The blisters are accessed via pressure actuation, a function provided by the analyzer/reader and embedded software, and thus are preferably foil blisters.

In certain embodiments, the cartridge comprises a bubble trap which allows for pressure relief, otherwise the fluid would not flow in the microfluidic channels. Alternatively, waste chambers can be closed under negative pressure and thus pull fluid in their direction when a valve is opened. In one embodiment, the cartridge comprises a reagent port, which can contain an absorbent pad having dried reagents thereon. Thus the reagent port can consist of an access hatch or affixed cover and a recess, into which a reagent pad can be placed. Alternatively, the reagent port could be a blister pack or an inlet allowing connection to external fluids. In certain embodiments, the cartridge comprises a waste reservoir and a waste reservoir external vent fluidly connected via a microfluidic channel to the assay chamber having a transparent access hatch or affixed cover allowing visual access to the chamber. The cartridge may also comprise a port to a waste chamber, although the chamber can be made sufficiently large to hold all waste and this port omitted.

The cartridges of the present invention can be made using any suitable method known in the art. The method of making may vary depending on the materials used. For example, devices substantially comprising a metal may be stamped, milled from a larger block of metal, or cast from molten metal. Likewise, components substantially comprising a plastic or polymer may be thermoformed, milled from a larger block, cast, or injection molded.

In certain embodiments, the cartridge is a disposable plastic chip made by injection molding and/or etching of parts and adhering layers together. Exemplary materials for constructing the cartridge are plastics of durometer 34-40 Shore D for the substrate and microfluidics, such as polymers and copolymers of styrene, acrylic, carbonate, butadiene, propylene, vinyl, acrylonitrile, and foil for the blisters. In some embodiments, the cartridge is made by 3D-printing or additive manufacturing techniques.

Some aspects of the present invention may be made using an additive manufacturing (AM) process. Among the most common forms of additive manufacturing are the various techniques that fall under the umbrella of “3D Printing”, including but not limited to stereolithography (SLA), digital light processing (DLP), fused deposition modelling (FDM), selective laser sintering (SLS), selective laser melting (SLM), electronic beam melting (EBM), and laminated object manufacturing (LOM). These methods variously “build” a three-dimensional physical model of a part, one layer at a time, providing significant efficiencies in rapid prototyping and small-batch manufacturing. AM also makes possible the manufacture of parts with features that conventional subtractive manufacturing techniques (for example CNC milling) are unable to create.

Suitable materials for use in AM processes include, but are not limited to, using materials including but not limited to nylon, polyethylene terephthalate (PET), acrylonitrile butadiene styrene (ABS), resin, polylactic acid (PLA), polystyrene, and the like. In some embodiments, an AM process may comprise building a three dimensional physical model from a single material, while in other embodiments, a single AM process may be configured to build the three dimensional physical model from more than one material at the same time.

In certain embodiments, the cartridge comprises one or more reagents (e.g. labeled detecting antibodies) for detection of biomarkers. For example, the bead sensor comprises a first antibody to capture a biomarker from the sample, while the cartridge comprises a second antibody (e.g. a labeled detecting antibody) that binds to a different epitope of the marker while bound to the first antibody of the bead sensor. The reagents may be within a blister pack or dried on a reagent pad.

In one embodiment, a reagent chamber is activated, allowing for a fluid or buffer to travel past a dry pad or chamber containing dried reagents (e.g., antibodies and stains), reconstitutes the same and carries these to the assay chamber. Optionally, these reagents can be premixed with the second chamber fluid. In one embodiment, the stability of antibody components is improved in the dry form. In one embodiment, the dry pads are exchangeable, e.g. via a hinged lid. The excess reagents can then be washed away, using wash from the first chamber, and the remaining signals detected and analyzed. In one embodiment, the dried reagents comprise one or more types of bioaffinity ligand. Additional assay chambers can be provided, depending on the number of analytes to be analyzed and the spectral range of the signals (and device capacity to distinguish same).

Further details of the cartridges may be found in U.S. Ser. Nos. 13/745,740, filed Jan. 18, 2013, Ser. No. 14/025,163, filed Sep. 12, 2013, Ser. No. 14/027,320, filed Sep. 16, 2013, Ser. No. 15/154,100, filed May 13, 2016, Ser. No. 15/658,730, filed Jul. 25, 2017, 61/484,492, filed May 10, 2011, and 61/558,165, filed Nov. 10, 2011, which are all expressly incorporated by reference herein in their entireties.

The cartridges may be constructed from common, inexpensive materials, including vinyl adhesive, laminate, stainless steel, and poly-(methyl methacrylate) (PMMA). Computer-aided design (CAD) models the cartridges, and then a CAD plotter/cutter incises the vinyl. Up to seven layers of vinyl/laminate are deposited on six to eight cartridges using conventional, parallel layering methods. In certain embodiments, cartridges are disposable and purposed to service one patient and a single assay. The cartridges may also be prepared from a three-layer plastic stack prepared by injection molded plastic methods. These three layers are sealed into a single coherent part using laser sealing procedures or various adhesive layers.

The agarose can be plain agarose, or any of the agarose derivatives such as cross-linked agarose, sepharose, or any agarose derivatives that can be used for affinity chromatography. The array can be on agarose beads or disk, as discussed above. Where disks are employed, the disk is preferably about 10-50 μm thick and 50-200 μm in width, but larger or smaller sizes are also possible, depending on sample size, specificity of the reagents, and the sensitivity of the instrumentation.

In one embodiment, the disk sits on a porous support or substrate, and the fluidics are such that fluid is forced through the disk. The porous substrate can be any membrane, such as nitrocellulose membrane, or poly(methyl methacrylate) (PMMA) membrane. It can also be a more substantive support, such as porous glass, ceramic, plastic (delrin, PMMA, acrylonitrile butadiene styrene, i.e. Abs), or metallic (e.g., stainless steel) frit. In one embodiment, the disk can sit in a well, and the fluids merely pass over the disk in the same way they would a bead. Where wells are used, either a plastic, glass, silicon, or stainless-steel chip arrayed with wells, each of which hosts an individual bead or disk sensor, may be used to complete the cartridge.

These arrays of antibodies can be easily exchanged, by substituting a new array on the cartridge, thus quickly and easily reprogramming the card for a new assay. The reprogramming can be completed, by uploading assay specific software to the analyzer device, via e.g., USB, and/or by providing different reagents and fluids in the blister packs or chambers or in dry reagent pads as needed.

In one embodiment, the cartridge comprises a detection or analysis window. In one embodiment, the analysis window can be covered with a transparent cover such as glass, polycarbonate, acrylic, and the like, under which is housed the array of agarose beads or disks. The cover is optional, particularly where the array is added by the user at the time of the test. However, if the array and cartridge are preassembled for sale, a cover can be beneficial as it prevents the array chip containing the agarose beads from getting dehydrated. The capture antibody conjugated beads are prepared in batches and are stored until use, with a demonstrated long-term stability. In one embodiment, a common detector antibody is contained in an upstream chamber in a dry form (e.g., in a dry porous pad) along with excipients to promote long term stability.

In one embodiment, a sample is applied to the cartridge via a specimen entry port, and the sample travels to the detection window where the arrayed capture antibodies capture the analyte of interest. Wash fluid (e.g., PBS or PBS plus detergent) from a blister pack on the cartridge is then activated, and travels to the array to wash away unbound sample. Next, PBS or other appropriate buffer is released and en route to the analysis window collects and reconstitutes the detection antibody, which will then stain the captured analytes on the beads or disks. Additional wash solution follows to wash off unbound detector antibody. A waste chamber downstream of the array collects all waste fluids leaving the array.

In one embodiment, purified calibration standards in the array are first analyzed to derive the standard curves to which tested clinical samples are compared. Dedicated image analysis algorithms convert fluorescent signals from the sample into quantitative measurements, through interpolation of signals developed from testing of samples on a dose curve generated from the purified calibration standards. These values are then used, together with any subject information that was inputted into the device to prepare and report a exposure and/or disease severity assessment.

Compared with gold standard systems, such as enzyme-linked immunoassay (ELISA), the p-BNC system has assay times measured in minutes rather than hours, limits of detection (LOD) two or more orders of magnitude lower, and multiplex capacity of 10 or more concurrent analytes with appropriate internal controls.

Biomarkers

In one aspect, the invention provides a systems and method for the diagnosis and management of patients having or at risk for having a pathogen-mediated disease or infection. For example, the system and method described herein can be used to quickly evaluate a subject as having or not having: a pathogen-mediated disease or infection. In some aspects, one or more of the biomarkers described herein are used to assess the presence of a pathogen (e.g., SARS CoV-2) in the subject or identify the subject as having a pathogen-mediated disease (e.g., COVID-19). In some aspects, one or more of the biomarkers described herein are used to assess disease severity of the pathogen-mediated disease or infection.

In one embodiment, the method comprises determining the level of one or more biomarkers in a biological sample and diagnosing a patient with COVID-19. In one embodiment, the one or more biomarkers are selected from the group consisting of: IgM, IgG, SARS CoV-2 spike. In one embodiment, the one or more biomarkers comprises an SARS CoV-2 antibody, for example an antibody that binds to a SARS CoV-2 antigen such as spike. In one embodiment, the one or more biomarkers are selected from the group consisting of a SARS-CoV-2 nucleocapsid protein and spike receptor binding domain (RBD) IgG antibody. In one embodiment, the method comprises determining the level of one or more biomarkers in a biological sample and assessing a subject as having a risk for developing a severe case of COVID-19. In one embodiment, the one or more biomarkers are selected from the group consisting of: CRP, PCT, CK-MB, c-TN-I, D-dimer, and NT-proBNP.

Biomarker tests provide key information about the health or disease status of an individual. In SARS CoV-2, the virus that causes COVID-19, the spike protein (S-protein) mediates receptor binding and membrane fusion. Spike protein contains two subunits, 51 and S2. S1 contains a receptor binding domain (RBD), which is responsible for recognizing and binding with the cell surface receptor. S2 subunit is the “stem” of the structure, which contains other basic elements needed for the membrane fusion. The spike protein is the common target for neutralizing antibodies and vaccines. It has been reported that SARS-CoV-2 (2019-nCoV) can infect the human respiratory epithelial cells through interaction with the human ACE2 receptor. Indeed, the recombinant Spike protein can bind with recombinant ACE2 protein. The Nucleocapsid Protein (N-protein) is the most abundant protein in coronavirus. The N-protein is a highly immunogenic phosphoprotein, and it is normally very conserved. The N protein of coronavirus is often used as a marker in diagnostic assays (Wang et al., 2003, Genomics Proteomics Bioinformatics, 1(2): 145-54).

For COVID-19, in analysis of 127 patients in Wuhan, China, the most common complications leading to death were acute cardiac injury (58.3%), ARDS (55.6%), coagulation dysfunction (38.9%), and acute kidney injury (33.3%) (Bai et al., 2020, Clinical and Laboratory Factors Predicting the Prognosis of Patients with COVID-19: An Analysis of 127 Patients in Wuhan, China (Feb. 26, 2020). Available at SSRN: https://ssrn.com/abstract=3546118). Death of patients was more likely to have multiple organ dysfunction syndrome (Bai et al., 2020, Clinical and Laboratory Factors Predicting the Prognosis of Patients with COVID-19: An Analysis of 127 Patients in Wuhan, China (Feb. 26, 2020). Available at SSRN: ssrn.com/abstract=3546118). Those patients that died from the infection had deteriorated at-admission liver and kidney function, tissue damage related biomarkers (lactate dehydrogenase, creatine kinase and troponin I), and prolonged prothrombin time. The inflammatory biomarkers, including C-reactive protein, are also significantly increased. Moreover, the prognostic values of troponin I and procalcitonin are found to be excellent (AUC=0.939 and =0.900, respectively). Further, regression model showed procalcitonin values ≥0.15 ng/ml serve as a key prognostic factor for death (Bai et al., 2020, Clinical and Laboratory Factors Predicting the Prognosis of Patients with COVID-19: An Analysis of 127 Patients in Wuhan, China (Feb. 26, 2020). Available at SSRN: https://ssrn.com/abstract=3546118). In another recent study, clinical data on 82 death cases laboratory-confirmed as SARS-CoV-2 infection, respiratory failure remained the leading cause of death (69.5%), following by sepsis syndrome/MOF (28.0%), cardiac failure (14.6%), hemorrhage (6.1%), and renal failure (3.7%). Furthermore, respiratory, cardiac, hemorrhage, hepatic, and renal damage were found in 100%, 89%, 80.5%, 78.0%, and 31.7% of patients, respectively. Most patients had a high neutrophil-to-lymphocyte ratio of >5 (94.5%), high systemic immune-inflammation index of >500 (89.2%), increased C-reactive protein level (100%), lactate dehydrogenase (93.2%), and D-dimer (97.1%) (Zhang et al., 2020, Clinical characteristics of 82 death cases with COVID-19. medRxiv 2020.02.26.20028191; doi: doi.org/10.1101/2020.02.26.20028191). Another study demonstrated that increasing odds of in-hospital death were associated with older age (odds ratio 1.10, 95% CI 1.03-1.17, per year increase; p=0.0043) and higher Sequential Organ Failure Assessment (SOFA) score (5.65, 2.61-12.23; p<0.0001). Most importantly, the study confirmed the importance of d-dimer as a prognostic factor with odds of in-hospital death significantly increased with d-dimer levels greater than 1 μg/mL (18.42, 2.64-128.55; p=0.0033) on admission (Zhou et al., 2020, Clinical course and risk factors for mortality of adult inpatients with COVID-19 in Wuhan, China: a retrospective cohort study. The Lancet, https://doi.org/10.1016/S0140-6736(20)30566-3). The severity of pneumonia, displayed by pulmonary hypertension, right ventricular pressure overload and the inflammatory cytokine response, as well as the presence of disease-relevant co-morbidities, namely heart failure and renal dysfunction, NT-proBNP, a marker of cardiac failure, has also been shown to be predictive of death in patients with community acquired pneumonia (Arram et al., 2013, Egyptian Journal of Chest Diseases and Tuberculosis, Volume 62, Issue 2, 2013 293-300).

Methods & Assays

In one embodiment, the invention provides a method for diagnosing, and assessing the severity of, a pathogen-mediated infection. In one embodiment, the invention provides a method for detecting COVID-19 and COVID-19 disease severity biomarkers in a biological sample.

In one embodiment, the invention provides a method for diagnosing COVID-19 in a subject. In one embodiment, the method provides community-wide disease surveillance and monitoring by diagnosing subjects, regardless of the presence of clinical signs or symptoms. Thus, in certain embodiments, the method comprises identifying subjects having COVID-19 and thus require isolation or quarantine. In certain embodiments, the method comprises identifying additional persons who should be tested based on their contact with the subject or other association with the subject.

In one embodiment, the invention provides a method for providing a prognosis for subject having a pathogen-mediated infection. For example, in some embodiments, the invention provides an assessment of COVID-19 disease severity. Thus, in some embodiments, the method can identify those subjects with COVID-19 who will likely need close monitoring, hospitalization, intensive care, ventilators, or therapeutic agents. Thus, in some embodiments, the method aids in allocating scarce resources among large number of subjects who test positive for having COVID-19.

In one embodiment, the invention provides a method of risk-stratification. For example, in one embodiment, the invention provides a method of decision-making of severity of COVID-19. In one embodiment, the method comprises selecting the acute management when the biomarker panel levels indicate a risk-stratification so that the subject may require hospitalization, use of a ventilator, or other specialized care.

In one embodiment, the method comprises: a) obtaining a biological sample from a patient; and b) testing said sample to determine the level of one or more biomarkers of a pathogen-mediated disease or infection or the disease severity of a pathogen-mediated disease or infection; wherein said testing is conducted using bioaffinity ligands specific for the biomarkers.

In one embodiment, the method comprises: a) obtaining a biological sample from a patient; and b) testing said sample to determine the level of one or more biomarkers of a pathogen-mediated disease or infection or the disease severity of a pathogen-mediated disease or infection; wherein said testing is conducted on an array of agarose beads, conjugated to bioaffinity ligands specific for the biomarkers, and wherein signal from said array of agarose beads is analyzed by circular area of interest or line profiling or both.

In one embodiment, the method comprises: a) obtaining a biological sample from a patient; and b) testing said sample to determine the level of one or more biomarkers of COVID-19 or the disease severity of COVID-19; wherein said testing is conducted using bioaffinity ligands specific for the biomarkers.

In one embodiment, the method comprises: a) obtaining a biological sample from a patient; and b) testing said sample to determine the level of one or more biomarkers of COVID-19 or the disease severity of COVID-19; wherein said testing is conducted on an array of agarose beads, conjugated to bioaffinity ligands specific for the biomarkers, and wherein signal from said array of agarose beads is analyzed by circular area of interest or line profiling or both.

In certain embodiments, the method comprises detecting the level of at least one, at least two, at least three, at least four, at least five, at least six, at least seven, at least eight, at least nine, at least ten, at least eleven, at least twelve, or at least thirteen of the biomarkers described herein.

In certain embodiments, the method comprises detecting the level of at least one, at least two, at least three, at least four, at least five, at least six, at least seven, at least eight, or at least nine of the biomarkers of: IgM, IgG, SARS CoV-2 spike, CRP, PCT, CK-MB, c-TN-I, D-dimer, and NT-proBNP. In one embodiment, the method comprises detecting one or more biomarkers comprising an SARS CoV-2 antibody, for example an antibody that binds to a SARS CoV-2 antigen such as spike. In one embodiment, the method comprises detecting the level of at least one biomarker selected from the group consisting of a SARS-CoV-2 nucleocapsid protein and spike receptor binding domain (RBD) IgG antibody.

In one embodiment, the method further comprises assigning a risk-stratification to the patient when the one or more biomarkers is above baseline level. In one embodiment, the baseline level is level of the one or more biomarkers in a sample from a non-diseased subject. In one embodiment, baseline level is a standard level of the one or more biomarkers. In one embodiment, the risk-stratification is a high, medium, or low. In one embodiment, the risk-stratification is a numerical score from 0-10. In one embodiment, the risk-stratification is a numerical score from 0-100. In one embodiment, the risk-stratification correlates to the risk of developing a severe, potentially fatal, case of the pathogen-mediated infection or disease (e.g., COVID-19).

In some embodiments, the method of assessing disease severity or assigning a risk-stratification to the patient includes accounting for one or more additional risk factors or demographic information of the patient, including but not limited to, age, gender, ethnicity, race, height, weight, body mass index (BMI), smoking status, and the presence of other medical conditions including but not limited to, cardiovascular disease, hypertension, hypercholesterolemia, prior stroke, prior myocardial infarction, lung disease, diabetes, renal failure, and liver disease. In some embodiments, an additional risk factor included in the present analysis is whether the patient is immunocompromised, for example as a result of cancer treatment. In some embodiments, an additional risk factor in the present analysis is whether the patient is severely obese [BMI >40]. In some embodiments, an additional risk factor in the present analysis is whether the patient's underlying medical condition, such as renal failure or liver disease, is not well controlled. In some embodiments, the method includes accounting for one or more clinical signs or symptoms from the patient, including, but not limited to, fever/body temperature, fatigue, coughing, nasal congestion, sore throat, diarrhea, vomiting, chest tightness, shortness of breath, and loss of consciousness.

In one embodiment, the method comprises a two-tiered model comprising a predictive algorithm (Tier-1) and a biomarker model (Tier-2). In one embodiment, Tier 1 uses non-laboratory data that are readily available prior to laboratory measurements and is intended to help determine whether Tier 2 biomarker-based testing and/or hospitalization are warranted. The Tier 2 Biomarker Model then predicts disease severity using biomarker measurements and patient characteristics. In one embodiment, the two-tiered model combines multiplex biomarker measurements and risk factors in a statistical learning algorithm to predict mortality with excellent diagnostic accuracy.

In one embodiment, Tier-1 accounts for one or more additional risk factors or demographic information of the patient, including but not limited to, age, gender, ethnicity, race, height, weight, body mass index (BMI), smoking status, and the presence of other medical conditions including but not limited to, cardiovascular disease, hypertension, hypercholesterolemia, prior stroke, prior myocardial infarction, lung disease, diabetes, renal failure, and liver disease. In some embodiments, an additional risk factor included in the present analysis is whether the patient is immunocompromised, for example as a result of cancer treatment. In some embodiments, an additional risk factor in the present analysis is whether the patient is severely obese [BMI >40]. In some embodiments, an additional risk factor in the present analysis is whether the patient's underlying medical condition, such as renal failure or liver disease, is not well controlled.

In some embodiments, the method includes accounting for one or more clinical signs or symptoms from the patient, including, but not limited to, fever/body temperature, fatigue, coughing, nasal congestion, sore throat, diarrhea, vomiting, chest tightness, shortness of breath, and loss of consciousness.

In one embodiment, Tier-1 may be used in any setting including but not limited to home care, primary care or urgent care clinics, emergency departments, hospital, and intensive care, etc.

In one embodiment, Tier-1 may further comprise a severity scoring system. In one embodiment, the severity scoring system may be used to measure risk factors in a statistical learning algorithm to predict mortality rate. In one embodiment, the severity scoring system may be used to predict severity of the disease and the need for ventilation or hospitalization. In one embodiment, patients may be treated differently based on their severity score. In one embodiment, patients with low severity score may be managed through a home or telemedicine setting. In one embodiment, patients with high severity score may be referred for a blood draw or biomarker based testing. In one embodiment, patients with high severity score may be hospitalized.

In one embodiment, Tier-1 may be used with symptomatic patients who are positive or presumably positive for COVID-19 and seeking care at a family health center or emergency room.

In one embodiment, Tier-1 may be easily tuned for high sensitivity or high specificity by adjusting the weighting or relative importance of sensitivity and specificity in clinical practice.

In one embodiment, Tier 2 comprises systems and methods for detection of a second panel of biomarkers to assess disease severity as described elsewhere herein. In some embodiments, the analysis may be performed using a hand-held device with disposable chip as described elsewhere herein. In one embodiment, the analysis may be done with any other device/method known to one skilled in the art.

In one embodiment, patients with low Tier 2 score may be managed in a low-to-moderate risk group (e.g., 5 day Telehealth follow-up). In one embodiment, patients with high Tier 2 score may be hospitalized in most cases or managed in a high risk group (e.g., 24-48 hour follow-up).

In one embodiment, the method comprises an improved COVID-19 screening system comprising a pre-screening algorithm and a point-of-care (POC) screening. In one embodiment, the method can be modified to be used in any medical facility including but not limited to a dental office. In one embodiment, the pre-screening algorithm helps determine if a patient is eligible for COVID-19 diagnostic testing (POC screening). In one embodiment, the pre-screening algorithm may use a combination of environmental, physiological, and demographic factors including but not limited to local positivity rate, case incidence rate, SpO2, temperature, ethnicity (Hispanic), and race (Asian, Black, White) to determine if a patient is eligible for COVID-19 diagnostic testing. In one embodiment, the pre-screening algorithm may use one or more of the environmental, physiological, and demographic factors to determine eligibility. In one embodiment, case incidence rate is calculated based on a state and county where the patient resides and the date of the encounter. In one embodiment, a web-based calculator then extracts the latest case incidence rate from public sources. In one embodiment, case incidence rate is calculated as the 7-day average cases per 100,000 within the specified state and county.

In one embodiment, the pre-screening test may be used to generate a score for a patient. In one embodiment, patients with high pre-screening score are recommended for the POC screening. In one embodiment, patients with low pre-screening score would be granted admission to a medical facility including but not limited to a dental office.

In one embodiment, the POC screening comprises: a) obtaining a biological sample from a patient; and b) assaying the sample for one or more antigens associated with SARS-CoV2 infection and one or more antibodies associated with SARS-CoV2 infection.

In one embodiment, POC screening may be performed in two sequential steps: a) antigen assay followed by b) antibody assay. In one embodiment, the rational of two step is that there is significant cross-reaction between capture and detecting reagents. In one embodiment, the first step delivers the anti-NP detecting antibody reagents and measures the antigen beads immunocomplex signal (anti-NP monoclonal+nucleocapsid protein+anti-NP polyclonal AF-488) while ignoring the antibody beads. In one embodiment, second step delivers the secondary anti-rabbit detecting antibody reagents and measures the antibody beads immunocomplex signal (RBD+2019-nCoV spike S1 antibody IgG+secondary anti-rabbit AF-488) in the panel while ignoring the antigen beads.

In certain embodiments, the method uses logistic regression, for example LASSO logistic regression, to transform the biomarker levels, demographic information, risk factors, and the like into a score that provides simple and relevant information to a user or health care provider regarding the presence of the pathogen and/or disease severity in the subject.

In one embodiment, the method further comprises performing an optimal clinical intervention. In one embodiment, the optimal clinical intervention is performed when the level of the one or more biomarkers are above a threshold level. Clinical management for hospitalized patients with COVID-19 is focused on supportive care of complications, including advanced organ support for respiratory failure, septic shock, and multi-organ failure. Empiric testing and treatment for other viral or bacterial etiologies may be warranted. Corticosteroids are not routinely recommended for viral pneumonia or ARDS and should be avoided unless they are indicated for another reason (e.g., COPD exacerbation, refractory septic shock following Surviving Sepsis Campaign Guidelines). There are currently no antiviral drugs licensed by the U.S. Food and Drug Administration (FDA) to treat COVID-19. Some in-vitro or in-vivo studies suggest potential therapeutic activity of some agents against related coronaviruses, but there are no available data from observational studies or randomized controlled trials in humans to support recommending any investigational therapeutics for patients with confirmed or suspected COVID-19 at this time. Remdesivir, an investigational antiviral drug, was reported to have in-vitro activity against COVID-19. A small number of patients with COVID-19 have received intravenous remdesivir for compassionate use outside of a clinical trial setting. A randomized placebo-controlled clinical trial of remdesivir for treatment of hospitalized patients with COVID-19 respiratory disease has been implemented in China. A randomized open label trial of combination lopinavir-ritonavir treatment has also been conducted in patients with COVID-19 in China, but no results are available to date (www.cdc.gov/coronavirus/2019-ncov/hcp/faq.html]

Biological samples can also be obtained from other sources known in the art, including whole blood, serum, plasma, urine, interstitial fluid, peritoneal fluid, cervical swab, tears, saliva, buccal swab, skin sample, and the like. In one embodiment, the biological sample is blood, saliva, plasma, or urine.

In one embodiment, the quantitative results generated will be utilized to train machine learning algorithms to provide an intuitive COVID19 ScoreCard.

In one embodiment, a method for training a machine learning algorithm comprises the steps of obtaining a quantity of biological samples from a plurality of subjects, including but not limited to whole blood, serum, plasma, urine, interstitial fluid, peritoneal fluid, cervical swab, tears, saliva, buccal swab, or skin, obtaining or calculating one or more biomarkers from the plurality of subjects, including but not limited to IgM, IgG, SARS CoV-2 spike, CRP, PCT, CK-MB, c-TN-I, D-dimer, and NT-proBNP, SARS-CoV-2 nucleocapsid protein and spike receptor binding domain (RBD) IgG antibody, obtaining one or more COVID-19 characteristics or outcomes from the plurality of subjects, and training a machine learning algorithm to optimize one or more predictive weighting coefficients of the biomarkers in order to build a predictive model. In certain aspects, the method further comprises obtaining a set of demographic data or other characteristics from the plurality of subjects and training the machine learning algorithm to optimize one or more predictive weighting coefficients of the biomarkers and/or demographic data in order to build a predictive model.

Aspects of the invention relate to a statistical learning algorithm, machine learning algorithm, machine learning engine, or neural network. A statistical learning algorithm may be trained based on various attributes of a subject for example one or more biomarkers described herein, and may output one or more predictive outcomes, diagnostic scorecard or prediction based on the attributes. In some embodiments, attributes may include biomarker measurements (cTnI, CK-MB, CRP, NT-proBNP, D-dimer, PCT, etc.), age, BMI, sex, smoking status, hypercholesterolemia, hypertension, previous stroke, previous myocardial infarction, and diabetes. The resulting predictive values, diagnostic values, or risk score may then be judged according to their success rate in matching one or more binary classifiers or quality metrics for known input values, and the weights of the attributes may be optimized to maximize the average success rate for binary classifiers or quality metrics. In this manner, a statistical learning algorithm can be trained to predict and optimize for any binary classifier or quality metric that can be experimentally measured. Examples of binary classifiers or quality metrics that a statistical learning algorithm can be trained on are discussed herein, including biomarker measurements (cTnI, CK-MB, CRP, NT-proBNP, D-dimer, PCT, etc.), age, BMI, sex, smoking status, hypercholesterolemia, hypertension, previous stroke, previous myocardial infarction, diabetes, and symptoms (fever/body temperature, fatigue, coughing, nasal congestion, sore throat, diarrhea, vomiting, chest tightness, shortness of breath, loss of consciousness. In some embodiments, the statistical learning algorithm may have multi-task functionality and allow for simultaneous prediction and optimization of multiple quality metrics.

In embodiments that implement such a neural network, a neural network of the present invention may identify one or more attributes whose predictive value (as evaluated by the neural network) has a high correlative value, thereby indicating a strong correlation with one or more results.

In some embodiments, the neural network may be updated by training the neural network using a value of the desirable parameter associated with an input biomarker values. Updating the neural network in this manner may improve the ability of the neural network in predictive accuracy of providing a disease severity or risk score. In some embodiments, training the neural network may include using a value of the desirable parameter associated with a known outcome. For example, in some embodiments, training the neural network may include predicting a value of a disease severity or risk score for a subject having a known patient outcome based on measured biomarkers, comparing the predicted value to the corresponding value associated with the known patient outcome, and training the neural network based on a result of the comparison. If the predicted value is the same or substantially similar to the observed value, then the neural network may be minimally updated or not updated at all. If the predicted value differs from that of the known score in view of the actual patient outcome, then the neural network may be substantially updated to better correct for this discrepancy. Regardless of how the neural network is retrained, the retrained neural network may be used to propose additional disease severity or risk scores.

Although the techniques of the present application are in the context of disease diagnosis, assessment, and treatment, it should be appreciated that this is a non-limiting application of these techniques as they can be applied to other types of parameters or attributes, for example, to facilitate epidemiological surveys of disease exposure, to assist patient triaging in resource-limited situations, and the like.

Depending on the type of data used to train the neural network, the neural network can be optimized for different types of diagnosis and treatment. Querying the neural network may include inputting an initial data set and set of one or more attributes disclosed herein. The neural network may have been previously trained using different data set. The query to the neural network may be for one or more predictive output values. A binary or non-binary output value may be received from the neural network in response to the query.

The techniques described herein associated with iteratively querying a neural network by inputting a training data set, receiving an output from the neural network that has one or more output values, and successively providing further data sets as an input to the neural network, can be applied to other machine learning applications. In some embodiments, an iterative process is formed by querying the neural network for one or more output parameters based on an input data set, receiving the one or more output parameters, and identifying one or more changes to be made to the input data set based on the output received. An additional iteration of the iterative process may include inputting the data set from an immediately prior iteration with one or more changes. The iterative process may stop when one or more output values substantially match the output values from a training iteration.

In one embodiment, the diagnostic or biomarker panel is a group of two or more, three or more, four or more, five or more, six or more, seven or more, 8 or more, or 9 or more biomarkers. In one embodiment, the diagnostic or biomarker panel correlates with the presence and/or severity of the pathogen-mediated infection or disease. In one embodiment, the subject is detected as having SARS CoV-2 and/or COVID-19 when one or more of IgM, IgG, SARS-CoV-2 nucleocapsid protein and spike receptor binding domain (RBD) IgG antibody and SARS CoV-2 spike is increased. In one embodiment, a subject is determined to have a high risk of having a severe case of COVID-19 when one or more of CRP, PCT, CK-MB, c-TN-I, D-dimer, and NT-proBNP is increased.

Assays & Kits

In one aspect, the invention provides an assay for determining the level of a biomarker of a pathogen-mediated infection or disease or a biomarker of the disease severity of the pathogen-mediated infection or disease. In one aspect, the invention provides an assay for diagnosing COVID-19 or the severity of COVID-19. In one embodiment, the assay comprises: a microfluidic lab-on-chip based immunoassay that comprises a disposable cartridge and a separate reader, wherein said cartridge fits into a slot on said reader, and said reader performs said immunoassay and outputs a result, wherein the cartridge comprises i) a generally flat substrate having embedded microfluidic channels connecting an inlet port to an embedded downstream assay chamber having a transparent cover and containing a removable array of bead sensors; ii) one or more reagent chambers fluidly connected to and upstream of said assay chamber; and iii) one or more waste fluid chambers fluidly connected to and downstream of said assay chamber; iv) wherein each bead sensor is a porous polymeric bead of size between 50-300 μm±10% having an antibody conjugated thereto, wherein said antibody specific to a biomarker. In one embodiment, wherein the immunoassay has a lower limit of detection for each of said biomarkers of <50 ng/ml and a detection range of at least four orders of magnitude. In one embodiment, cartridge comprises 2 or more, 3 or more, 4 or more, 5 or more, 6 or more, 7 or more, 8 or more, or 9 or more of the antibodies.

In one embodiment, the invention provides a kit for diagnosing a pathogen-mediated infection or disease or assessing disease severity of the pathogen mediated infection or disease. In one embodiment, the invention provides a kit for diagnosing COVID-19 or assessing COVID-19 disease severity. In one embodiment, the kit comprises a cartridge of the invention. In one embodiment, the cartridge is wrapped in an airtight package. In one embodiment, the kit further comprises a vial of assay fluid. The kit can include other components, e.g., instructions for use.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

ScoreCard Analysis

The multiplexing capacity of the technology is important for all aspects of care related, including diagnosis, prognosis, monitoring, risk stratification and guidance for therapeutic interventions of patients. As such, these dedicated efforts in a single setting results in the creation of a new diagnostic COVID-19 assessment tool based on a multiplexed panel of biomarkers, the ScoreCard, as described herein.

Clinical decision support systems (CDSSs) are support tools which assist in medical decision-making by providing clinicians with personalized assessments or recommendations and offer a promising solution for managing and diagnosing COVID-19. CDSSs have been developed, featuring various machine-learning methods such as artificial neural networks, Support Vector Machines, random forest, Bayesian networks, logistic regression, and ensemble methods. Although CDSSs promise enhanced diagnostic results, shorter wait times, and reduced cost versus the standard of care, physicians may be hesitant to implement “black box” CDSSs (i.e., the algorithm's results and methods to obtain them are either uninterpretable or not capable of providing actionable therapeutic recommendations). Therefore, the ScoreCard uses a lasso logistic regression approach, converting risk factors and biomarker data into a single score with interpretable and clinically useful information in the form of logistic regression coefficients.

Fashioned as a sensor that learns, these bead-based smart sensors were found to be an excellent tool for capturing and detecting soluble analytes (McRae, 2016, Accounts of Chemical Research, 49(7): 1359-6810). This platform was applied for drug testing, testing for cardiac and inflammation biomarkers, and allergy testing (Christodoulides et al., 2015, Drug and Alcohol Depend, 153: 306-313; Christodoulides et al., 2005, LOC, 5(3): 261-269; Christodoulides et al., 2012, Method. DeBakey Cardiovas J, 8(1): 6-12). The Cardiac ScoreCard is a clinical decision support system uses LASSO logistic regression to transform multiple risk factors and biomarker measurements into a one score with intuitive and clinically relevant information. The Cardiac ScoreCard provides personalized reports for a range of CVDs with diagnostic and prognostic models for cardiac wellness, acute myocardial infarction, and heart failure. The cardiac scorecard was developed using data obtained from a prospective NIH sponsored trial involving 1050 recruited patients at two clinical sites. A total of 15 biomarkers (including all those biomarkers targeted here for COVID 19) were measured across serum and saliva samples en route to development of a series of high performance multivariate diagnostic models. Similarly, best in class precision lesion diagnostic models and an effective adjunct technology has been developed and validated through another prospective NIH sponsored trial involving 999 patients (McRae, 2016, Exp. Syst. With Applic, 54: 136-147).

Additional information regarding certain aspects of the system, method, or device described herein, can be found in U.S. Pat. No. 8,257,967, WO03090605, US20060073585, US2006079000, US2006234209, WO2004009840, WO2004072097, U.S. Pat. Nos. 7,781,226, 8,101,431, 8,105,849, US2006257854, US20060257941, US2006257991, WO2005083423, WO2005085796, WO2005085854, WO2005085855, WO2005090983, U.S. Pat. No. 8,377,398, WO2007053186, US2010291431, WO2007002480, US2008050830, WO2007134191, US2008038738, WO2007134189, US2008176253, US2008300798, WO2008131039, US2012208715, WO2011022628, US2013130933, WO2012021714, US2013295580, WO2012065117, US2013274136, WO2012065025, WO2012154306, US2012322682, US20130295580, US20140235487, US20140094391, US20150111778, each of which are incorporated by reference in their entireties.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples, therefore, specifically point out the preferred embodiments of the present invention, and are not to be construed as limiting in any way the remainder of the disclosure.

### Example 1

Managing COVID-19 with a Clinical Decision Support Tool in a Community Health Network: Algorithm Development and Validation

An integrated point-of-care COVID-19 Severity Score and CDSS has been developed which combines multiplex biomarker measurements and risk factors in a statistical learning algorithm to predict mortality with excellent diagnostic accuracy (McRae M P, et al., 2020, Lab Chip. 20(12):2075-85). The COVID-19 Severity Score was trained and evaluated using data from 160 hospitalized COVID-19 patients from Wuhan, China. The COVID-19 Severity Scores were significantly higher for patients who died as compared with patients who were discharged with median (interquartile range [IQR]) scores of 59 (40-83) and 9 (6-17), respectively, and area under the curve (AUC) of 0.94 (95% confidence interval [CI] 0.89-0.99).

The COVID-19 condition has caused and continues to cause significant morbidity and mortality globally. A validated tool to assess and quantify viral sepsis severity and patient mortality risk would address the urgent need for disease severity categorization. The unfolding novel COVID-19 pandemic has greatly illuminated the important role of community health centers in providing safe and effective patient care. This invention describes a clinical decision support tool for COVID-19 disease severity developed using recent data from the Family Health Centers (FHC) and externally validated using data from two recent studies from hospitals in Wuhan, China. A practical and efficient tiered approach is described which involves a model with non-laboratory inputs (Tier 1), a model with biomarkers commonly measured in ambulatory settings (Tier 2), and a mobile app to deliver and scale these tools. The deployment of these new capabilities has potential for immediate clinical impact in community clinics whereby such tools could lead to improvements in patient outcomes and prognostic judgment.

The materials and methods employed in these experiments are now described.

Patient Data

Data from 701 patients with COVID-19 were collected across 9 clinics and hospitals within the FHC network at NYU Langone, one of the largest Federally Qualified Health Center networks in the US. All patients had detectable SARS-CoV-2 infection by polymerase chain reaction (PCR) testing. The following outcomes were recorded: not hospitalized, discharged, ventilated, and deceased. Validation data for the Tier 1 Outpatient Model were derived from a study of 160 hospitalized COVID-19 patients from Zhongnan Hospital of Wuhan University. Validation data for the Tier 2 Biomarker Model were derived from a study of 375 hospitalized COVID-19 patients from Tongji Hospital in Wuhan, China (Yan L. et al., 2020. Nat. Mach. Intell. 2(5):283-8).

Clinical Decision Support Tool

This invention describes the development of a 2-tiered CDSS for the assessment of COVID-19 disease severity, using similar methods as described previously (McRae, M P et al., 2020, Lab. Chip. 20(12):2075-85; McRae, M P et al., 2016, Expert Sys. Appl. 54:136-47). The Tier 1 Outpatient Model uses non-laboratory data that are readily available prior to laboratory measurements and is intended to help determine whether Tier 2 biomarker-based testing and/or hospitalization is necessary. Here, a lasso logistic regression model was trained to distinguish between patients that were not hospitalized or were hospitalized and discharged home without need for ventilation versus patients that were ventilated or died. Patients who were still hospitalized when the data were compiled were excluded. The following predictors were considered in model training: age, gender, body mass index, systolic blood pressure, temperature, symptoms (cough, fever, or shortness of breath), known cardiovascular comorbidities (patient problem list includes one or more of cerebrovascular disease, heart failure, ischemic heart disease, myocardial infarction, peripheral vascular disease, and hypertension), pulmonary comorbidities (asthma and chronic obstructive pulmonary disease), and diabetes.

The Tier 2 Biomarker Model predicts disease severity using biomarker measurements and patient characteristics. A lasso logistic regression model was trained to distinguish patients that died versus patients that were either never hospitalized or discharged home. Patients who were ventilated and/or still hospitalized when the data were compiled were excluded. The following predictors were considered in model training: age, gender, comorbidities, C-reactive protein (CRP), cardiac troponin I (cTnI), D-dimer, procalcitonin (PCT), and N-terminal pro-B-type natriuretic peptide (NT-proBNP). Predictors that were not relevant to the model (i.e., coefficients equal to zero) were removed. Laboratory measurements across all time points were log-transformed. Patients with no measurements for the aforementioned biomarkers were excluded. Biomarker values below the limits of detection were set to the minimum measured value divided by the square root of 2.

Model Development and Statistical Analysis

Both Tier 1 and 2 models were developed using the same procedure. All continuous predictors were standardized with mean of zero and variance of one. Missing data were imputed using the multivariate imputation by chained equations algorithm in statistical software R (Buuren, S. et al., 2011, J. Stat. Software 45(3):1-67). Predictive mean matching and logistic regression imputation models were used to generate 10 imputations for continuous and categorical predictors, respectively. Samples in the training and test sets were partitioned using stratified 5-fold cross-validation to preserve the relative proportions of outcomes in each fold. Model training and selection were performed on each of the 10 imputation datasets for 10 Monte Carlo repetitions and optimized for the penalty parameter corresponding to one standard error above the minimum deviance for additional shrinkage. After initial training, only predictors with nonzero regression coefficients were retained, and the model was retrained with a reduced number of predictors. The training process was repeated until all predictors yielded nonzero coefficients. Model performance was documented in terms of mean (95% CI) of AUC, sensitivity, specificity, positive predictive value (PPV), and negative predictive value (NPV). Median (IQR) cross-validated COVID-19 Scores were compared across disease outcomes. The COVID-19 Scores for both models and biomarker measurements were compared using Wilcoxon rank sum test. Normally distributed predictors were compared using an independent t-test. Proportions were compared using the Chi-squared test (Campbell I., 2007, Stat. Med. 26(19):3661-75; Richardson, J T E, 2011, Stat. in Med. 30(8):890). Two-sided tests were considered statistically significant for P<0.05.

External Validation

The Tier 1 Outpatient Model was externally validated using data from a study of 160 hospitalized COVID-19 patients from Zhongnan Hospital of Wuhan University. Only patients with complete information (age, systolic blood pressure, gender, diabetes, and cardiovascular comorbidities) were included. Model performance was documented in terms of AUC, sensitivity, specificity, PPV, and NPV. Results were presented in a scatter/box plot of COVID-19 Outpatient Scores on patients that were discharged and those that died.

Similarly, the Tier 2 Biomarker Model was externally validated using data from a study of 375 hospitalized COVID-19 patients from Tongji Hospital in Wuhan, China collected between January 10 and Feb. 18, 2020 (Yan, L et al., 2020, Nat. Mach. Intell.,2(5):283-8). While most patients had multiple lab measurements over time, the first available lab value for each biomarker was used to validate the model to maximize lead time. Patients with one or more missing predictor values were excluded. Model performance was documented in terms of AUC, sensitivity, specificity, PPV, and NPV. Results were presented in a scatter/box plot of COVID-19 Biomarker Scores on patients that were discharged and those that died.

To demonstrate how the COVID-19 Biomarker Score could be used to track changes in disease severity over time, the model was evaluated on time series biomarker data. Since lab measurements were reported asynchronously, the model was reevaluated every time a new biomarker measurement became available. Time series plots of the COVID-19 Biomarker Score were generated for each patient.

The results of these experiments are now described.

The development of a 2-tiered CDSS to assess COVID-19 disease severity is described using similar methods as described previously (McRae, M P et al., 2020, Lab. Chip. 20(12):2075-85; McRae, M P et al., 2016, Experts Sys. Appl. 54:136-47). The Tier 1 Outpatient Model uses non-laboratory data that are readily available prior to laboratory measurements and is intended to help determine whether Tier 2 biomarker-based testing and/or hospitalization are warranted. The Tier 2 Biomarker Model predicts disease severity using biomarker measurements and patient characteristics.

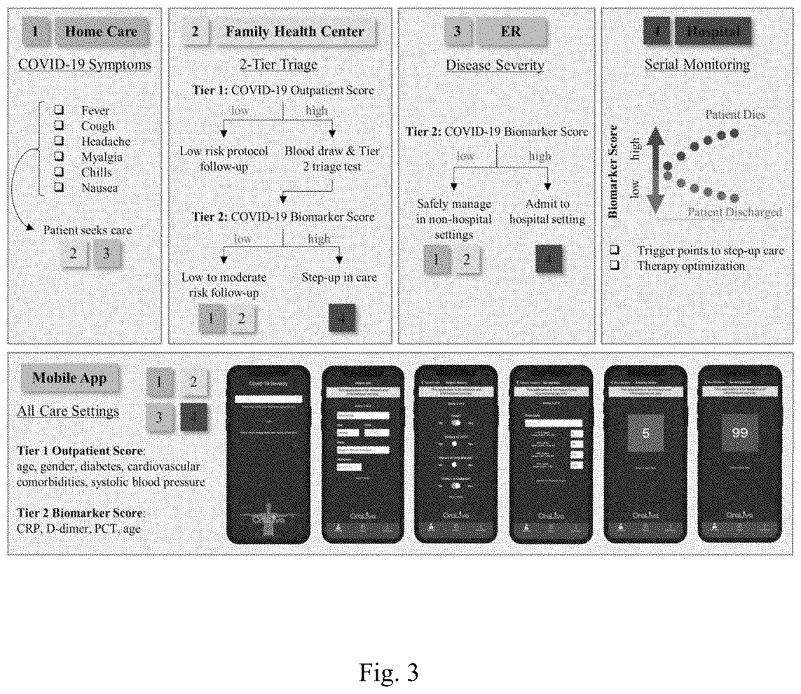

The CDSS and mobile app are designed to support decisions made in multiple settings, including (1) home care, (2) primary care or urgent care clinics, (3) emergency departments, and (4) hospital and intensive care (FIG. 3). The process starts with symptomatic patients who are positive or presumably positive for COVID-19 and seeking care at a family health center or emergency room. In the family health center, decisions are made in two key stages, or tiers. The Tier 1 algorithm is intended for individuals in an outpatient setting where laboratory data are not yet readily available, (e.g., age, gender, blood pressure, and comorbidities). Patients with a low COVID-19 Outpatient Score may be managed through a home or telemedicine setting, while patients with a high COVID-19 Outpatient Score are referred for a blood draw and Tier 2 biomarker-based test. The Tier 2 algorithm, which is directly related to mortality risk, predicts disease severity using biomarker measurements and age. Patients with a low COVID-19 Biomarker Score are expected to be managed in a low-to-moderate risk group (e.g., 5 day Telehealth follow-up), while patients with a high COVID-19 Biomarker Score are expected to be hospitalized in most cases or managed in a high risk group (e.g., 24-48 hour follow-up). Providers encountering clinically evident severe cases, as in urgent care or emergency departments, may choose to bypass the Tier 1 Outpatient Score and perform biomarker testing and Tier 2 triage on all COVID-19 patients. Lastly, in the hospital setting, patients are serially monitored for their COVID-19 Biomarker Score. Such personalized time series information directly related to mortality risk has strong potential to optimize therapy, improve patient care, and ultimately save lives. For both algorithms, cutoffs were selected that balanced sensitivity and specificity; however, these algorithms can be easily tuned for high sensitivity or high specificity by adjusting the weighting or relative importance of sensitivity and specificity in clinical practice.

Out of the 701 patients with detectable COVID-19 infection cared for by one of the 9 clinics within the FHC network, 402 were not hospitalized, 185 were hospitalized and discharged, 19 were ventilated, and 95 died (Table 1). Ventilated and deceased patients were older than those that were not hospitalized or discharged (P=0.03 and <0.001, respectively). Males accounted for 74% and 63% of patients who were ventilated and deceased vs. 46% for patients with less severe disease (i.e., not hospitalized or discharged) (P=0.02 and 0.002, respectively). Diabetes was also a statistically significant factor with 47% and 55% in the ventilated and deceased groups vs. 25% in the non-hospitalized and discharged groups (P=0.03 and <0.001, respectively). Likewise, 53% of ventilated patients (P=0.04) and 68% of deceased patients (P<0.001) had one or more cardiovascular comorbidities vs. 31% for the less severe disease categories, with hypertension being the most common. Interestingly, systolic blood pressure was significantly higher for patients who were not hospitalized vs. those that were discharged (P=0.004), and patients who died had abnormally low blood pressure relative to less severe disease (P<0.001). All biomarkers (cTnI, CRP, PCT, D-dimer, and NT-proBNP) were measured at significantly higher levels in patients that died vs. those that were not hospitalized or discharged (P<0.001).

TABLE 1
Characteristics of patients included in model training. Data are
represented as n (%), mean ± standard deviation, or median (IQR).

| | Not hospitalized | | Discharged | | Ventilated | | Deceased | |
|---|---|---|---|---|---|---|---|---|
| | n = 402 | | n = 185 | | n = 19 | | n = 95 | |
| Age, years | 48 ± 17 | | 50 ± 17 | | 58 ± 20 | | 67 ± 14 | |
| Gender | 182 | (45) | 89 | (48) | 14 | (74) | 60 | (63) |
| BMI^{a}, kg/m² | 25 ± 4 | | 28 ± 6 | | 29 ± 5 | | 25 ± 6 | |
| Systolic BP^{b}, | 132 ± 14 | | 123 ± 19 | | 126 ± 20 | | 94 ± 40 | |
| mmHg | | | | | | | | |
| Diastolic BP^{b}, | 82 ± 8 | | 71 ± 11 | | 70 ± 12 | | 54 ± 26 | |
| mmHg | | | | | | | | |
| Temperature | 99 ± 1 | | 98 ± 5 | | 99 ± 1 | | 100 ± 2 | |
| Pulse, beats per | 90 ± 18 | | 84 ± 14 | | 93 ± 14 | | 74 ± 54 | |
| min. | | | | | | | | |
| Asthma | 44 | (11) | 12 | (6) | 3 | (16) | 6 | (6) |
| COPD^{c} | 60 | (15) | 17 | (9) | 3 | (16) | 15 | (16) |
| Cancer | 13 | (3) | 5 | (3) | 2 | (11) | 14 | (15) |
| Cardiovascular | 120 | (30) | 61 | (33) | 10 | (53) | 65 | (68) |
| comorbidities^{d} | | | | | | | | |
| Diabetes | 96 | (24) | 53 | (29) | 9 | (47) | 52 | (55) |
| HIV/AIDS | 3 | (1) | 2 | (1) | 0 | (0) | 3 | (3) |
| Liver disease | 11 | (3) | 10 | (5) | 2 | (11) | 4 | (4) |
| Renal disease | 20 | (5) | 17 | (9) | 3 | (16) | 21 | (22) |
| cTnI, pg/mL | 7.07 | (7.07-7.07) | 7.07 | (7.07-7.07) | 20.00 | (7.07-63.75) | 73.50 | (7.07-712.00) |
| CRP, mg/L | 51.40 | (16.55-101.35) | 67.90 | (17.95-121.50) | 37.30 | (27.30-139.72) | 176.00 | (115.00-287.00) |
| PCT, ng/mL | 0.12 | (0.06-0.36) | 0.10 | (0.05-0.31) | 0.69 | (0.07-1.91) | 1.61 | (0.35-8.31) |
| D-Dimer, μg/mL | 0.39 | (0.20-0.71) | 0.27 | (0.18-0.56) | 0.86 | (0.50-3.02) | 1.58 | (0.72-5.35) |
| NT-proBNP, | 93.00 | (36.50-375.25) | 88.00 | (28.50-298.00) | 217.00 | (78.00-394.25) | 937.00 | (160.25-5728.50) |
| pg/mL | | | | | | | | |

^{a}BMI: body mass index
^{b}BP: blood pressure
^{c}COPD: chronic obstructive pulmonary disease
^{d}Cardiovascular comorbidities: one or more of cerebrovascular disease, heart failure, ischemic heart disease, myocardial infarction, peripheral vascular disease, and hypertension

Tier 1 Outpatient Model

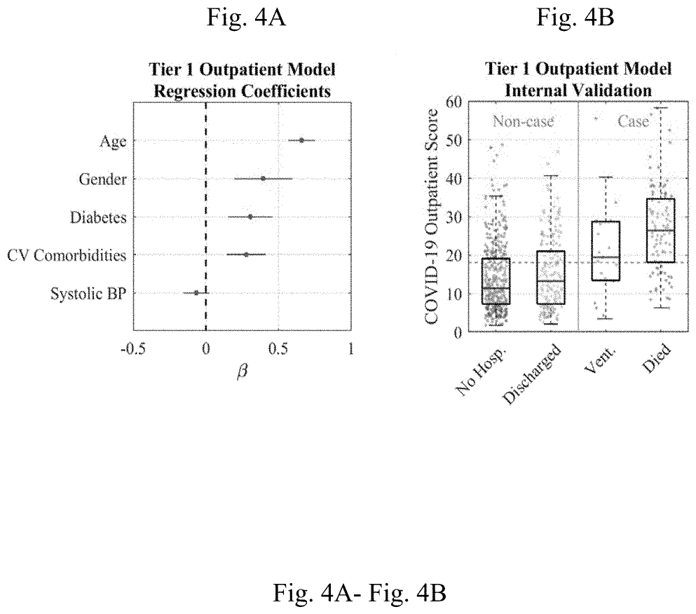

The Tier 1 Outpatient Model for COVID-19 disease severity was developed and internally validated using data from the FHCs at NYU Langone (FIG. 4A-FIG. 4B). The model retained the following predictors: age, gender, systolic blood pressure, cardiovascular comorbidities (one or more of cerebrovascular disease, heart failure, ischemic heart disease, myocardial infarction, peripheral vascular disease, and hypertension), and diabetes. Median COVID-19 Outpatient Scores were 11, 13, 20, and 27 for not hospitalized, discharged, ventilated, and deceased patients, respectively. The model's AUC (95% CI) was 0.79 (0.74-0.84) at the optimal cutoff COVID-19 Outpatient Score of 18 (Table 2). Median scores (FIG. 4A-FIG. 4B) had statistically significant differences for comparisons between all patient groups except not hospitalized vs. discharged (P=0.18).

TABLE 2
Internal validation performance in terms of AUC, sensitivity,
specificity, PPV, and NPV (95% CI) from 5-fold cross-
validation. Tier 1 and 2 models were trained and tested
using data from FHCs at NYU.

| | |
|---|---|
| Tier 1 Outpatient | Tier 2 Biomarker |
| Model | Model |

| | | | | |
|---|---|---|---|---|
| AUC | 0.79 | (0.74-0.84) | 0.95 | (0.92-0.98) |
| Sensitivity | 0.73 | (0.69-0.76) | 0.89 | (0.86-0.92) |
| Specificity | 0.73 | (0.69-0.76) | 0.89 | (0.86-0.92) |
| PPV | 0.34 | (0.30-0.38) | 0.70 | (0.65-0.74) |
| NPV | 0.93 | (0.91-0.95) | 0.97 | (0.94-0.98) |

Tier 2 Biomarker Model

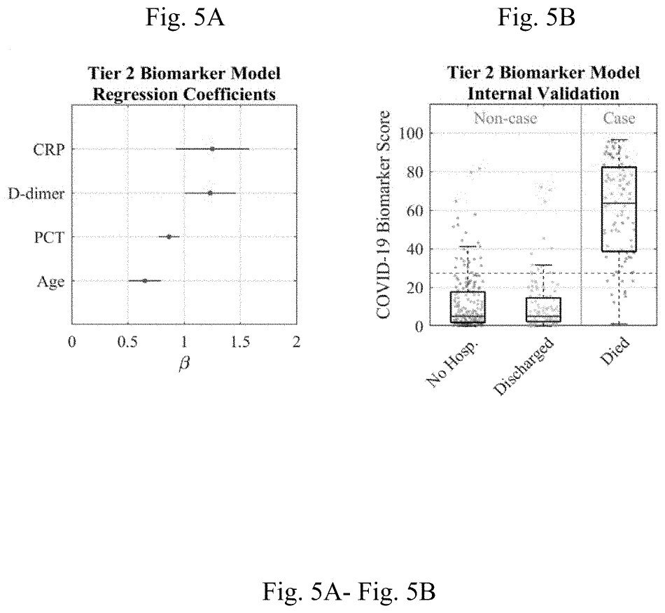

The Tier 2 Biomarker Model for COVID-19 disease severity was developed and internally validated using data from the FHCs at NYU Langone (FIG. 5A-FIG. 5B). Patients who were ventilated (n=19) and still hospitalized (n=19) were excluded. Patients with fewer than one biomarker measurement were excluded (n=190 not hospitalized, n=64 discharged, n=1 deceased). The remaining 427 patients with one or more biomarker measurement were included in the analysis (n=212 not hospitalized, n=121 discharged, n=94 deceased). The model retained the following predictors after shrinkage and selection: age, D-dimer, PCT, and CRP. Median COVID-19 Outpatient Scores were 5, 5, and 64 for not hospitalized, discharged, and deceased patients, respectively. The model's AUC (95% CI) was 0.95 (0.92-0.98) at the optimal cutoff COVID-19 Outpatient Score of 27 (Table 2). Median COVID-19 Outpatient Scores (FIG. 5A-FIG. 5B) had statistically significant differences for comparisons between not hospitalized vs. died (P<0.001) and discharged vs. died (P<0.001).

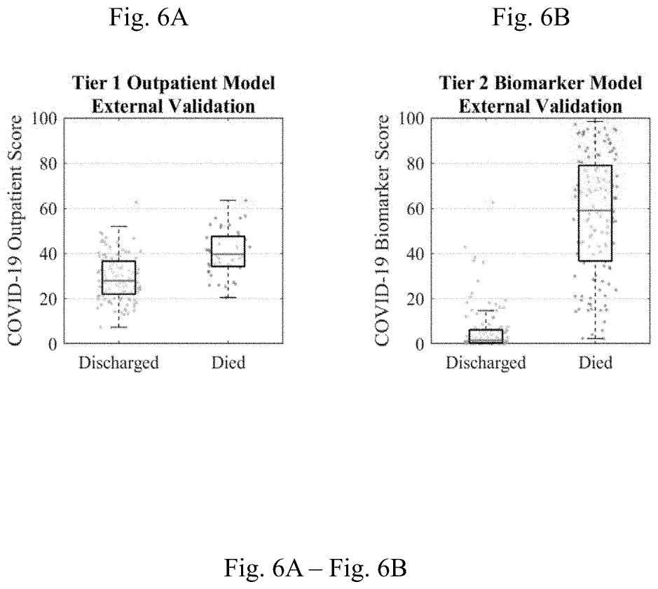

External Validation The Tier 1 Outpatient Model was externally validated using data from a study of 160 hospitalized COVID-19 patients with hypertension from Zhongnan Hospital of Wuhan University, Wuhan, China (Guo, T et al., 2020, JAMA Cardiol.). Out of the 160 patients in the study, 4 were missing one or more predictors and were excluded from the analysis. The COVID-19 Biomarker Scores were evaluated for 115 patients who were discharged and 41 patients who died (FIG. 6A). The median (IQR) COVID-19 Biomarker Scores were 27.9 (22.0-36.4) for patients that were discharged and 39.7 (34.2-47.4) for patients that died. The external validation diagnostic performance was determined using a cutoff score of 34 (Table 3).

The Tier 2 Biomarker Model were externally validated using data from a study of 375 hospitalized COVID-19 patients from Tongji Hospital in Wuhan, China collected between Jan. 10 and Feb. 18, 2020 (Yan L. et al., 2020. Nat. Mach. Intell. 2(5):283-8). In order to maximize potential lead time, the first available laboratory measurements during hospitalization were used to generate cross-sectional COVID-19 Biomarker Scores, representing the first in a series of measurements collected for hospital stays lasting a median (IQR) of 12.5 (8-17.5) days prior to the outcomes (discharged or deceased). Out of the 375 patients in the study, 133 were missing one or more lab value and excluded from the analysis. The COVID-19 Biomarker Scores were evaluated for 112 patients who were discharged and 130 patients who died (FIG. 6B). The median (IQR) COVID-19 Biomarker Scores were 1.6 (0.5-6.2) for patients that were discharged and 59.1 (36.6-78.9) for patients that died. The external validation diagnostic performance was determined using a cutoff score of 19 (Table 3).

TABLE 3
External validation performance in terms of AUC, sensitivity,
specificity, PPV, and NPV (95% CI). The Tier 1 Outpatient
Model was evaluated on Zhongnan Hospital dataset [26],
The Tier 2 model evaluated on Tongji Hospital dataset (Yan
L. et al., 2020. Nat. Mach. Intell. 2(5): 283-8).

| | |
|---|---|
| Tier 1 Outpatient | Tier 2 Biomarker |
| Model | Model |

| | | | | |
|---|---|---|---|---|
| AUC | 0.79 | (0.70-0.88) | 0.97 | (0.95-0.99) |
| Sensitivity | 0.76 | (0.68-0.82) | 0.89 | (0.84-0.93) |
| Specificity | 0.73 | (0.65-0.80) | 0.93 | (0.89-0.96) |
| PPV | 0.50 | (0.42-0.58) | 0.94 | (0.90-0.96) |
| NPV | 0.89 | (0.83-0.94) | 0.88 | (0.83-0.92) |

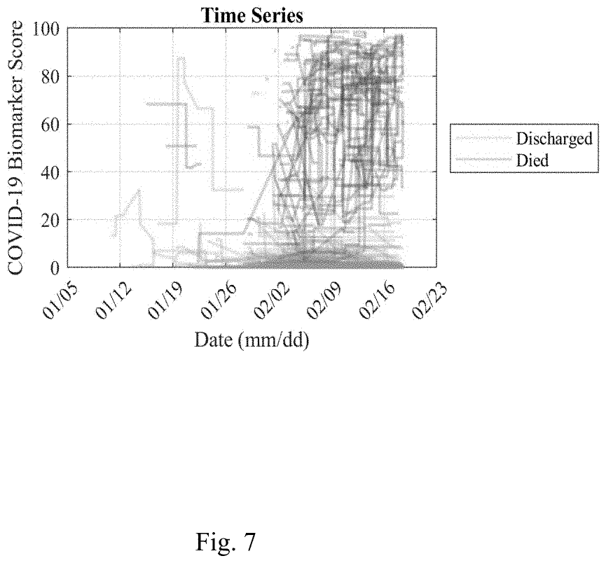

The COVID-19 Biomarker Scores were also evaluated for patients over time using longitudinal biomarker measurement data from individual patients in the external validation set (FIG. 7). When comparing the first scores after admission vs. the final measurements prior to discharge/death, patients who recovered and were discharged had an average decrease in score of 4.7 while patients who died had an average increase in score of 11.2.

As the COVID-19 pandemic continues to create surges and resurgences without an effective vaccine, the goal of this multidisciplinary team was to develop a triage and prognostication tool that strengthens community-level testing and disease severity monitoring. A CDSS and mobile app for COVID-19 disease severity have been designed, developed, and validated using data from 1236 patients with COVID-19 across numerous clinics and hospitals in the coronavirus disease epicenters of Wuhan, China and New York, USA. These clinically validated tools have potential to assist healthcare providers in making evidence-based decisions in managing COVID-19 patient care. The significance of this work is realized by the algorithms developed and validated here, which are accurate, interpretable, generalizable.

With respect to accuracy, both Tier 1 and Tier 2 models were effective at discriminating disease outcomes with statistically significant differences between the most relevant patient groups (AUCs of 0.79 and 0.97 for Tier 1 and Tier 2 external validation, respectively). As expected, the Tier 1 Outpatient Model diagnostic accuracy in terms of AUC was lower than Tier 2 Biomarker Model, which demonstrates the importance of biomarker data in determining disease severity. Accurately identifying patients with elevated risk for developing severe COVID-19 complications can empower healthcare providers to save lives by prioritizing critical care, medical resources, and therapies.

Another strength of this approach is the interpretability of the models. While many predictive tools rely on ‘black box’ methods in which algorithmic decisions and the logic supporting those decisions are uninterpretable, the lasso logistic regression method is transparent through its coefficients (i.e., log odds) and probabilistic output. The Tier 1 Outpatient Score is the probability of severe disease (ventilation or death) based on the predictors (age, gender, diabetes, cardiovascular comorbidities, and systolic blood pressure). Likewise, the Tier 2 Biomarker Score is the probability of mortality based on CRP, D-dimer, PCT, age. Predictive models such as these are more likely to be adopted for clinical applications which value transparency and interpretability.

One of the most clinically relevant features of this new CDSS is the capacity to monitor individual patients over time. The use of this precision diagnostic approach allows for the amplification of early signs of disease that can be achieved by focusing on time-course changes of biomarker signatures that are referenced not to population metrics, but rather back to the individual patient. As an example, the use of time course changes in individual biomarker fingerprints has been explored previously in the study of early detection in ovarian cancer (Skates, S J et al., 2001, J. Am. Stat. Assoc. 96(454):429-39). Studies demonstrated that CA-125 by itself for a single time point was a poor diagnostic marker due to overlapping reference range problems across the population. However, when each patient was treated as their own point of reference and biomarker slopes for individual patients were considered, the diagnostic accuracy for this same biomarker increased significantly. Similarly, the COVID-19 Biomarker Score time series (FIG. 7) reveals a strong capacity to separate patients who die of COVID-19 complications from those who are discharged from the hospital. Note that the app includes capabilities to use the proximal biomarker measurements allowing for biomarker measurements to be collected over time without the rigid restriction of having all biomarker measurements be completed at the same time for all time points. This flexibility is anticipated to afford more convenience for longitudinal monitoring of patients.

Lastly, the models developed here demonstrated generalizability through external model validation. External validation is essential before implementing prediction models in clinical practice (Bleeker, S E et al., 2003, J Clin Epidemiol. 56(9):826-32). It was found that the AUCs for both Tier 1 and Tier 2 models were similar for internal vs. external validation, demonstrating that the models are generalizable to making predictions for these disease indications despite different care settings and patient demographics. Usually, prediction models perform better on the training data than on new data; however, in this study, it was found that the external validation results were approximately the same or better (Tier 1: AUC of 0.79 vs. 0.79; Tier 2: 0.95 and 0.97 for internal and external validation, respectively), suggesting that patients in the external validation sets may have suffered from more severe disease.

Despite the potential for CDSSs to transform health care, major challenges remain for translating and scaling such tools. Future data and, thus, model performance may have large heterogeneity, which is exacerbated by missing data (potentially not missing at random), non-standard definitions of outcomes, and incomplete laboratory measurements and follow-up times (Riley, R D et al., 2016, BMJ 353:1-11). The mobile app developed here is intended to reduce heterogeneity by encouraging the harmonization of data collection across multiple care settings. Further, models may be tuned through optimization of cutoffs for certain patient subpopulations. Another challenge in deploying a CDSS that relies on biomarker measurements is accounting for differences in laboratory testing across hospitals and clinics. The variability of such measurements across institutions may have a large impact on the distribution of COVID-19 Biomarker Scores. This challenge creates a unique opportunity for standardized, well-calibrated, and highly scalable point-of-care tests for COVID-19 disease severity (McRae M P et al., 2020, Lab. Chip. 20(12):2075-85; McRae, M P et al., 2016, Acc Chem Res. 49(7):1359-68; McRae, M P et al., 2015, Lab Chip. 15(20):4020-31).

A commercial app has been developed for deployment of these tools to frontline healthcare workers managing COVID-19 patients. The usability, user satisfaction, and confidence is being assessed in results of this CDSS and mobile app in the FHCs at NYU. This assessment focus on point-of-care testing capabilities to more rapidly assess the Tier 2 Biomarkers described in this study using a previously developed and published platform (McRae M P et al., 2020, Lab. Chip. 20(12):2075-85; McRae, M P et al., 2016, Acc Chem Res. 49(7):1359-68; McRae, M P et al., 2015, Lab Chip. 15(20):4020-31). The deployment of these new capabilities has potential for immediate clinical impact in community clinics, where the application of such tools could significantly improve the quality of care.

### Example 2

Integrated AI and Point-of-Care Solutions for COVID-19 Screening

Close proximity to patients and frequent potential for viral exposure through aerosol-generating procedures makes dentistry one of the highest risk occupations amid the COVID-19 pandemic. With asymptomatic and presymptomatic cases serving as the main driving force for community spread, there remains concern that screening patients upon entry for symptoms and temperature may be inadequate to detect subclinical infection. Improved screening and diagnostic testing are critical to tracing and breaking the chain of transmission. The goal of this study is to develop an improved COVID-19 screening system, comprising predictive algorithms and point-of-care (POC) testing, that is appropriate for dental settings. A retrospective analysis of 2553 pre- and asymptomatic patients who were tested for SARS-CoV-2 by RT-PCR was conducted.

Pre-screening algorithms were developed to determine whether proceeding to a diagnostic test is necessary. Further, a proof-of-concept combination COVID-19 antigen/antibody test was developed on a POC platform. The full pre-screening model had an AUC (CI) of 0.76 (0.73-0.78). Despite being the default method for screening, temperature had lower AUC (0.52 [0.49-0.55]) compared to case incidence rate (0.65 [0.62-0.68]) and local positivity rate (0.71 [0.67-0.73]). POC assays for SARS-CoV-2 nucleocapsid protein and spike receptor binding domain (RBD) IgG antibody showed promising preliminary results, demonstrating a convenient, rapid (15-20 mins), quantitative, and sensitive (ng/mL) antigen/antibody assay. For pre-screening, time- and location-specific community spread data, such as case incidence and positivity rates, were more accurate in predicting COVID-19 status in patients without symptoms. Subsequent combination antigen/antibody approaches may significantly improve the accuracy of COVID-19 screening/diagnosis, including asymptomatic and subclinical infections, helping address unmet needs in dental settings.

As COVID-19 continues to spread uncontrollably around the world (World Health Organization 2020), dental communities continue to face enormous challenges in providing their services safely amid the pandemic. According to the US Department of Labor, several professions with the highest risk of contracting SARS-CoV-2 are in the dental field (dental hygienists, oral and maxillofacial surgeons, dental assistants, and dentists) due to close proximity to patients and high viral loads in the oral, nasal, pharyngeal mucosa, and respiratory secretions (Mahmud, P K et al., 2020). Further, dental and anesthesia-based practices commonly use aerosol-generating procedures and frequently encounter unpredictable reflexes, such as gagging and coughing (Chanpong, B et al., 2020, Anesth. Prog. 67(3):127-134; Gupta, J et al., 2009, Indoor Air. 19(6):517-525). Containment measures adopted to reduce the spread of COVID-19 (eg, social distancing, self-isolation, travel restrictions) have resulted in a reduced workforce across many economic sectors (Nicola, Metal., 2020, Int J Surg. 78:185-193), especially for dental practices—many of which were temporarily forced to close, except for emergent care, by state mandates. As a result, some practices permanently closed and many experienced significant financial loss (Consolo, U et al., 2020, Int. J. Environ. Res. Public Health. 17(10):3459; Gasparro, R et al., 2020, Int. J. Environ. Res. Public Health. 17(15)). Currently, the only tools widely available to minimize transmission risk in dental offices are personal protective equipment, disinfection, and aerosol mitigation protocols. Likewise, screening patients upon entry for symptoms and temperature has not been shown definitively to detect those with subclinical infection (Letizia, A G et al., 2020, New England. Journal of Medicine 383.25: 2407-16). With asymptomatic and presymptomatic cases serving as a driving force for the community spread of COVID-19 (Ra, S H et al., 2021, Thorax 76.1: 61-63), diagnostic testing is critical to tracing and breaking the chain of transmission. Dental practices would benefit greatly from office-based point-of-care (POC) tests, ideally using sputum, saliva, and/or finger-stick blood samples (The Testing for Tomorrow Collaborative 2020).

Real-time reverse transcriptase polymerase chain reaction (RT-PCR) is the current gold standard method for SARS-CoV-2 detection. While this method has excellent sensitivity, results are usually reported within hours or days and requires specialized laboratories and highly trained technicians, making the methodology unsuitable for POC dental office screening. Although potentially less sensitive than RT-PCR, rapid (˜15 minute) and inexpensive immunoassays for SARS-CoV-2 antigen seek out specific proteins (eg, spike protein, hemagglutinin esterase protein, viral envelope) found in the virus and are deemed more appropriate for POC use. Whereas molecular diagnostic tests like RT-PCR and antigen tests can only reveal whether a person is currently infected with SARS-CoV-2, antibody tests detect the body's immune response to viral exposure which can persist in the bloodstream for many months after infection. About 80% of COVID-19 patients will eventually develop symptoms (Buitrago-Garcia, D et al., 2020, PLOS Med. 17(9):e1003346). In dental office settings, these symptomatic patients can easily be identified and have procedures rescheduled. However, asymptomatic or presymptomatic patients are much more challenging to identify and pose a major transmission risk. Pre-admission or pre-procedure diagnostic testing may be used to identify those with subclinical infection and further reduce exposure risk, but very few tests have met a high standard for sensitivity and specificity (Burger, D, 2020, ADA News). In a recent study, RT-PCR was reported to have a 66.7% detection rate whereas total antibodies testing had a 38.3% detection rate within the first week of infection (Zhao, J et al., 2020, Clin Infect Dis. 71(16):2027-34). However, combining the results from RT-PCR and IgM enzyme-linked immunosorbent assay (ELISA) allowed for a 98.6% detection rate within the first 5.5 days post-infection (Guo, L et al., 2020, Clin. Infect. Dis. 71(15):778-85). Rapid POC testing for combination SARS-CoV-2 virus and antibodies could detect patients with subclinical infections more effectively. Such tools with enhanced diagnostic accuracy could be used chairside with potential to have a dramatic influence on the dental industry alongside safe management of the spread of COVID-19 moving forward.

It is clear that POC tests are becoming crucial in identifying infected individuals to ensure they are isolated from the general population. While these kits are not currently available for widespread use, public and private organizations worldwide are working on prototypes, with over 50 currently in development (Kubina, R et al., 2020, Diagnostics. 10(6):434). To date, these new diagnostic tests have been developed outside of an integrated screening procedure. The development and customization of diagnostic tests tailored for the dental community is a key priority alongside its use with gated patient screening and risk-based triage procedures. None of the existing diagnostic tests cover both the initial screening process as well as comprehensive POC diagnostic testing for those patients with elevated risks of infection.

Over the past few years, diagnostic tools suitable for dental settings, including a platform to digitize biology with the capacity to learn (McRae, 1VIP et al., 2016, Acc. Chem. Res. 49(7):1359-1368), a POC oral cytopathology tool for assessment of potentially malignant oral lesions (McRae, 1VIP et al., 2020, Cancer Cytopathology. 128(3):207-20), and novel cytological signatures, such as nuclear F-actin, detected on the same platform (McRae et al., 2020, Journal of dental research: 0022034520973162) have been developed. There is also a history of developing saliva-based tests on the same platform (Christodoulides, N et al., 2015, Drug Alcohol Depend. 153:306; Christodoulides, N et al., 2005, Lab Chip. 5(3):261-69). In the past months, a general framework for implementing a POC clinical decision support system (McRae, M P et al. 2016, Expert Syst. Appl. 54:136-147) was published, which was adapted to the task of predicting mortality in cardiac patients with COVID-19 (McRae, M P et al., 2020, J. Med. Internet Res. 22(8):e22033). More recently, a two-tiered system for evaluating COVID-19 prognosis in inpatient and outpatient settings was developed using data from a diverse population of patients across the New York City metropolitan area and externally validated using data from hospitals in Wuhan, China (McRae, M P et al., 2020, Lab Chip. 20(12):2075-2085). In this study, whether pre-screening patients using convenient non-laboratory data can predict COVID-19 status in patients without symptoms is explored. This invention also introduces a POC solution for COVID-19 screening suitable for use in dental offices that has potential to be used in conjunction with the newly developed pre-screening method here reported. This integrated diagnostic includes a combination SARS-CoV-2 antigen and antibody (IgG) saliva test, covering the entire diagnostic timeline of the disease with a single multiplexed test. A preliminary assay validation was performed for this duplex COVID-19 antigen/antibody test.

The materials and methods employed in these experiments are now described.

Patient data

Pre-screening algorithms were developed from a retrospective analysis of asymptomatic or presymptomatic patient encounters resulting in a COVID-19 RT-PCR test. Data were collected across clinics and hospitals within the Family Health Centers (FHC) network at New York University Langone Health from Jan. 1 to Jun. 25, 2020. Data were analyzed at the encounter level rather than the patient level, because many patients had multiple encounters. Symptomatic patient encounters, in which one or more primary COVID-19 symptoms (cough, fever, shortness of breath) was present, were excluded. Physiological predictors were evaluated at two levels (systolic blood pressure <120 mmHg, diastolic blood pressure <80 mmHg, body temperature ≥99° F., pulse rate <80 bpm, oxygen saturation ≤96%). County-level testing data was acquired from the New.

York State Department of Health (New York State Statewide COVID-19 Testing 2020). For each patient, a local positivity rate was calculated (i.e., the average test positivity rate within the county of the reporting health center from 8 days to 1 day prior to the patient encounter). Similarly, case incidence rate was calculated as the local 7-day average cases per 100,000.

Model development and statistical analysis

Pre-screening models were developed using similar procedures described in an earlier publication (McRae M P et al., 2020, J. Med. Internet Res. 22(8):e22033). A lasso logistic regression model was trained to distinguish between asymptomatic or presymptomatic patient encounters that resulted in a positive vs. negative result for SARS-CoV-2 by RT-PCR. Continuous predictors were standardized with mean of zero and variance of one. Missing data were imputed using the multivariate imputation by chained equations package in statistical software R (Buuren, S et al., 2011, J. Stat. Softw. 45(3)). Samples in the training and test sets were partitioned and trained using stratified 5-fold cross-validation. Model cutoffs were selected to obtain at least 90% sensitivity. Diagnostic performance was documented in terms of mean area under the curve (AUC), sensitivity, specificity, positive predictive value (PPV), and negative predictive value (NPV). Normally distributed predictors were compared using an independent t-test. Proportions were compared using the Chi-squared test (Campbell, I., 2007, Stat. Med. 26(19):3661-3675; Richardson, J T E, 2011, Stat. Med. 30(8):890). Two-sided tests were considered statistically significant for P<0.05.

COVID-19 antigen/antibody assay development

The quantitative POC antigen/antibody combination test was developed for the detection of SARS-CoV-2 nucleocapsid protein and spike receptor binding domain (RBD) IgG antibody. In-house fabricated agarose beads sensors, with potential to host a variety of proteins and molecules, were utilized as the backbone for assay chemistry. The anti-nucleocapsid protein monoclonal antibody (Sino Biological #40143-R019) was conjugated in-house to the agarose bead sensors, as was recombinant RBD protein. The RBD was produced in Expi293F cells transfected with the vector pCAGGS SARS-CoV-2 RBD (BEI Resources #NR-52309) following the methods of Stadlbauer et al., 2020, but using PEI as the transfection reagent then supplementing the media with valproic acid as per Fang et al. 2017, Biol. Proceed. Online. 19:11-11. Anti-nucleocapsid protein polyclonal antibody (Sino Biological #40588-T62) was conjugated in-house to a fluorescent tag (Alexa Fluor 488 conjugation labelling kit, Invitrogen #A20181), and a secondary anti-rabbit antibody (Invitrogen) was also procured. Antigen (2019-nCoV nucleocapsid His recombinant protein, Sino Biological #40588-VO8B) and antibody (2019-nCoV spike 51 antibody IgG, Sino Biological #40150-R007) assessments were made in PBS (Thermo Fisher Scientific). A 10% (w/v) bovine serum albumin (BSA) (Sigma-Aldrich) solution was used for reagent stability, blocking nonspecific binding, and was used as sample carrier spiked in a dose-dependent manner with the analytes.

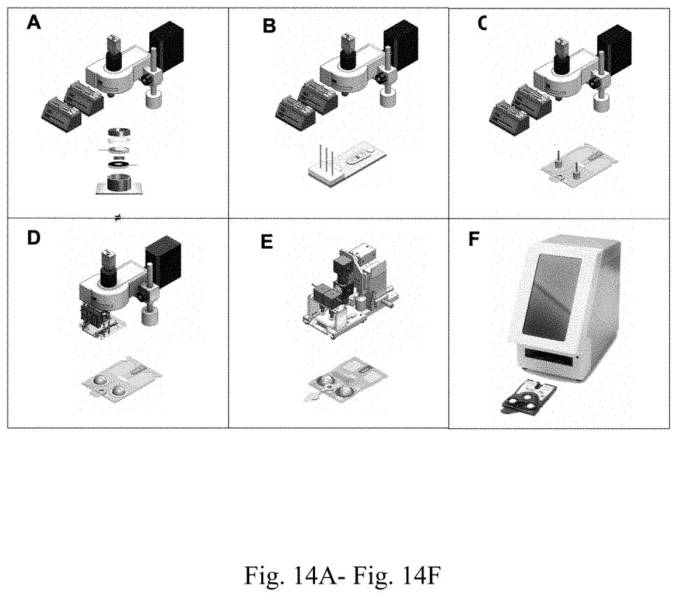

Assays were performed using prototype microfluidic cartridges, non-form factor instrumentation (see FIG. 14A-FIG. 14F), and software described previously (McRae, M P et al., 2015, Lab Chip. 15(20):4020-4031). Analyte-specific beads were deposited into the cartridge, allowing multiple measurements on the same assay. The 16-minute assay was performed at room temperature under continuous flow (PBS). Bead sensor priming, sample delivery, reagent incubation, wash steps, and image collection were completed using an Olympus fluorescent microscope and syringe pumps. Standard curves for both assays were completed using spiked samples (0, 2.4, 10, 40, 160, 625, 2500, 2500, and 10 000 ng/mL) and fit to 5-parameter logistic regression. Limit-of-detection (LOD) values were calculated using blank control replicates (average signal intensity plus 3 standard deviations).

The results of these experiments are now described.

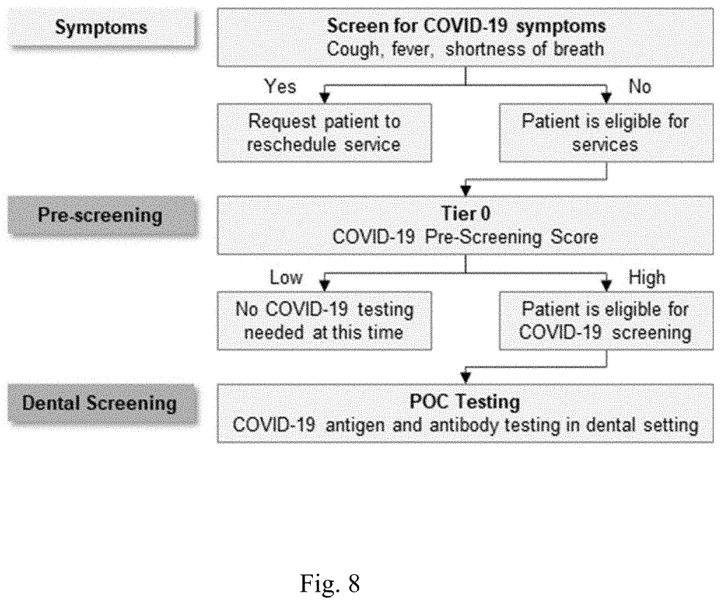

This current study encompasses the development of an integrated COVID-19 screening capability for dental settings that fits within the scope of a larger multi-tiered clinical decision support ecosystem to assess the entire disease spectrum of COVID-19 in multiple care settings (FIG. 8) (McRae, M P et al. 2020, J. Med. Internet Res. 22(8):e22033). The process starts with patients seeking dental care. Prior to entering the dental office, patients may be screened for the presence of one or more symptoms (fever, cough, and shortness of breath) of COVID-19. If symptomatic, patients should be requested to reschedule their appointments for a later date. All remaining patients without symptoms may then be pre-screened according to the pre-screening algorithm. Patients with pre-screening scores above the high-risk threshold may then be eligible for the POC COVID-19 antigen/antibody test. Patients testing negative for COVID-19 antigen/antibodies may proceed with dental procedures, while those testing positive may be requested to reschedule procedures and recommended for confirmation via RT-PCR testing.

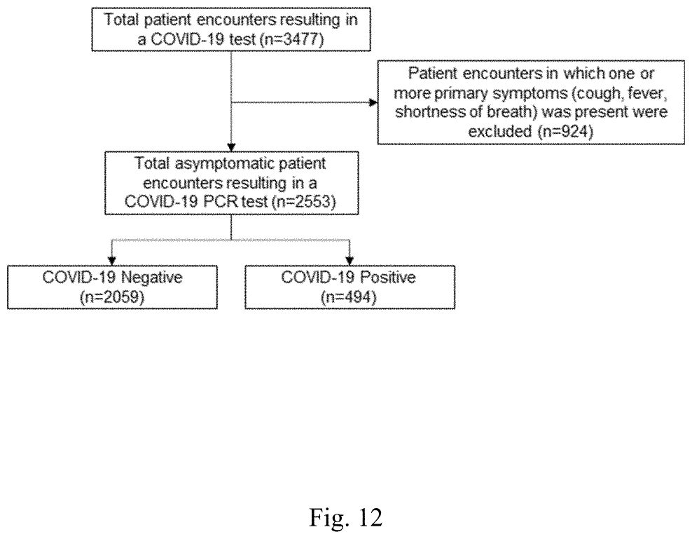

A retrospective analysis of non-laboratory data was studied to determine whether pre-screening patients could effectively rule out COVID-19 negative patients (i.e., to reduce the number of unnecessary tests). Given the nature of how pre-screening would be implemented in practice and that many patients had multiple encounters, we performed our analysis according to encounters rather than at the patient level. A total of 3477 patient encounters resulting in a SARS-CoV-2 RT-PCR test at NYU Langone Health FHCs were considered for analysis. Patient encounters with one or more primary symptoms (cough, fever, shortness of breath) were excluded (n=924 encounters). The remaining 2553 asymptomatic or presymptomatic patient encounters had either tested negative (n=2059 encounters) or positive (n=494 encounters) for SARS-CoV-2 by RT-PCR (FIG. 12).

Table 4 shows the characteristics of the study population at the patient and encounter levels. A total of 1074 asymptomatic or presymptomatic patients across 2553 encounters were tested for SARS-CoV-2 via RT-PCR testing. Comparing patients who tested positive vs. negative, age, gender, and body mass index (BMI) were not statistically significant factors (P=0.443, 0.883, and 0.130, respectively). With respect to race, Whites and Asians accounted for a smaller proportion of the asymptomatic or presymptomatic positives relative to those testing negative (P=0.005 and 0.021). Those with Hispanic ethnicity accounted for 56.6% of the positives vs. 38.7% negatives (P<0.001). While comorbid conditions may play a role in the severity of disease for those with COVID-19, none of the conditions studied had significantly different proportions in those that tested positive vs. negative. At the patient encounter level, all physiological measurements had statistically significant differences in proportions between RT-PCR positive and negative groups at their respective cutoffs (all P<0.05). The local positivity rate was significantly higher for those testing positive (32.8%) vs. negative (17.7%) (P<0.001). Similarly, the local case incidence rate was higher for COVID-19 positives vs. negatives (30.1 vs. 21.4 cases per 100 000, P<0.001).

TABLE 4
Characteristics of asymptomatic or presymptomatic patients resulting in a RT-PCR
test for SARS-CoV-2 at NYU Langone Health's FHCs. Data are represented as n (%)
or mean ± standard deviation. COPD = chronic obstructive pulmonary disease.
SpO2 = oxygen saturation. Local positivity rate is the 7-day average test
positivity in the county where the patient is receiving care. Local case incidence
rate is the 7-day average case incidence in the county where the patient is receiving care.

| | RT-PCR Negative | | RT-PCR Positive | | P-value |
|---|---|---|---|---|---|
| Patient-level | | | | | |
| No. of patients | 770 | | 304 | | |
| Encounters per patient | 1.3 ± 0.6 | | 1.2 ± 0.5 | | 0.015 |
| Age | 48 ± 17 | | 47 ± 17 | | 0.443 |
| Gender (no. of males) | 280 | (36.4) | 112 | (36.8) | 0.883 |
| BMI | 29.3 ± 7.9 | | 27.9 ± 5.3 | | 0.130 |
| Race | | | | | |
| White | 298 | (38.7) | 90 | (29.6) | 0.005 |
| Black | 137 | (17.8) | 44 | (14.5) | 0.191 |
| Asian | 77 | (10.0) | 17 | (5.6) | 0.021 |
| Other | 258 | (33.5) | 153 | (50.3) | <.001 |
| Ethnicity - Hispanic | 298 | (38.7) | 172 | (56.6) | <.001 |
| Cardiac comorbidities | 218 | (28.3) | 73 | (24.0) | 0.154 |
| Hypertension | 186 | (24.2) | 70 | (23.0) | 0.696 |
| Peripheral vascular | 83 | (10.8) | 23 | (7.6) | 0.112 |
| disease | | | | | |
| Heart failure | 38 | (4.9) | 11 | (3.6) | 0.352 |
| Cerebrovascular | 30 | (3.9) | 14 | (4.6) | 0.598 |
| disease | | | | | |
| Myocardial infarction | 21 | (2.7) | 8 | (2.6) | 0.931 |
| Ischemic heart disease | 8 | (1.0) | 6 | (2.0) | 0.224 |
| Asthma | 81 | (10.5) | 24 | (7.9) | 0.192 |
| Cancer | 49 | (6.4) | 18 | (5.9) | 0.787 |
| COPD | 104 | (13.5) | 30 | (9.9) | 0.104 |
| Diabetes | 116 | (15.1) | 49 | (16.1) | 0.666 |
| HIV/AIDS | 4 | (0.5) | 3 | (1.0) | 0.391 |
| Liver disease | 30 | (3.9) | 12 | (3.9) | 0.969 |
| Renal disease | 35 | (4.5) | 13 | (4.3) | 0.848 |
| Encounter-level | | | | | |
| No. of encounters | 2059 | | 494 | | |
| Systolic blood pressure <120 mmHg | 270 | (13.1) | 141 | (28.5) | <.001 |
| Diastolic blood pressure <80 mmHg | 426 | (20.7) | 186 | (37.7) | <.001 |
| Temperature ≥99° F. | 47 | (2.3) | 29 | (5.9) | <.001 |
| Pulse <80 bpm | 251 | (12.2) | 87 | (17.6) | 0.001 |
| SpO2 ≤96% | 105 | (5.1) | 74 | (15.0) | <.001 |

| | | | |
|---|---|---|---|
| Local Positivity Rate (%) | 17.7 ± 17.6 | 32.8 ± 20.1 | <.001 |
| Local Case Incidence | 21.4 ± 15.8 | 30.1 ± 16.2 | <.001 |
| Rate (cases per 100 000) | | | |

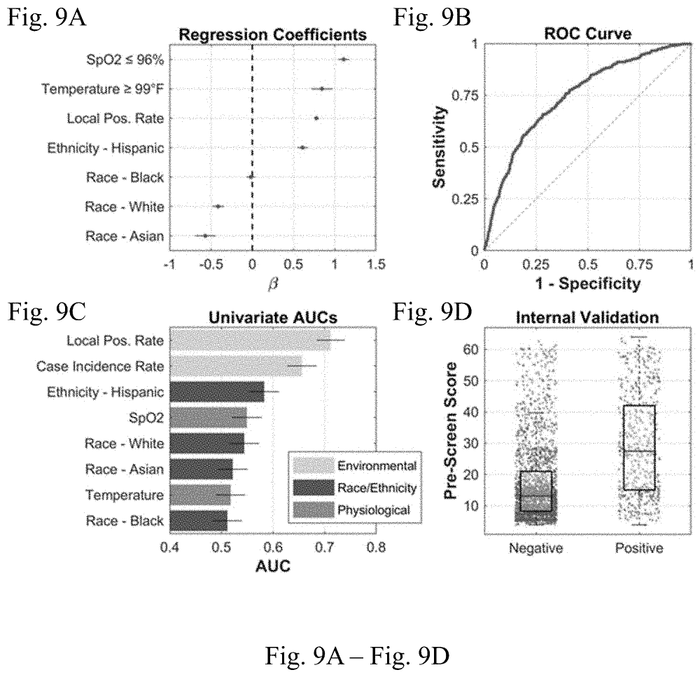

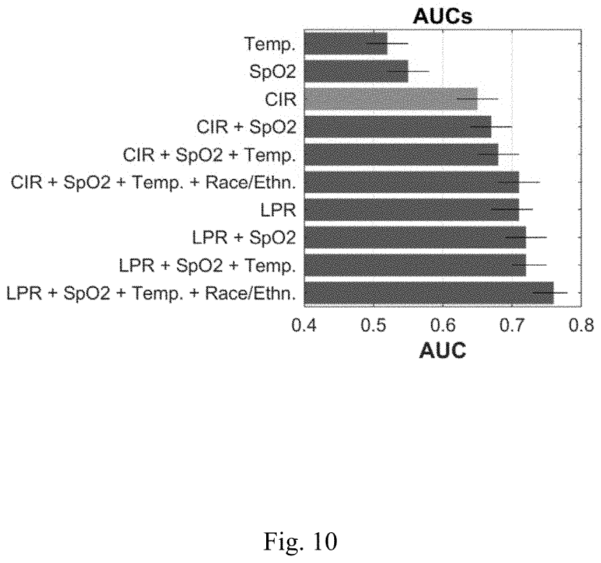

Pre-screening models for COVID-19 were developed and internally validated (FIG. 9A-FIG. 9D and Table 6). In the full model comprising local positivity rate, SpO2, temperature, ethnicity (Hispanic), and race (Asian, Black, White), the local test positivity rate was the most discriminatory individual predictor (univariate AUC 0.71 [0.68-0.74]). The full model, which combined environmental, physiological, and demographic factors, had an AUC of 0.76 (0.73-0.78). Median (IQR) COVID-19 pre-screening scores were 12 (8-22) and 28 (15-44) for negative and positive patients, respectively. FIG. 10 shows various diagnostic models that were developed to demonstrate the incremental effect of adding predictors. Despite being the default method for screening in dental settings, a model with only temperature had lower AUC (0.52 [0.49-0.55]) compared to all other models, including case incidence rate (0.65 [0.62-0.68]), and local positivity rate (0.71 [0.67-0.73]). The preferred model (case incidence rate only) had an AUC of 0.65 (0.62-0.68).

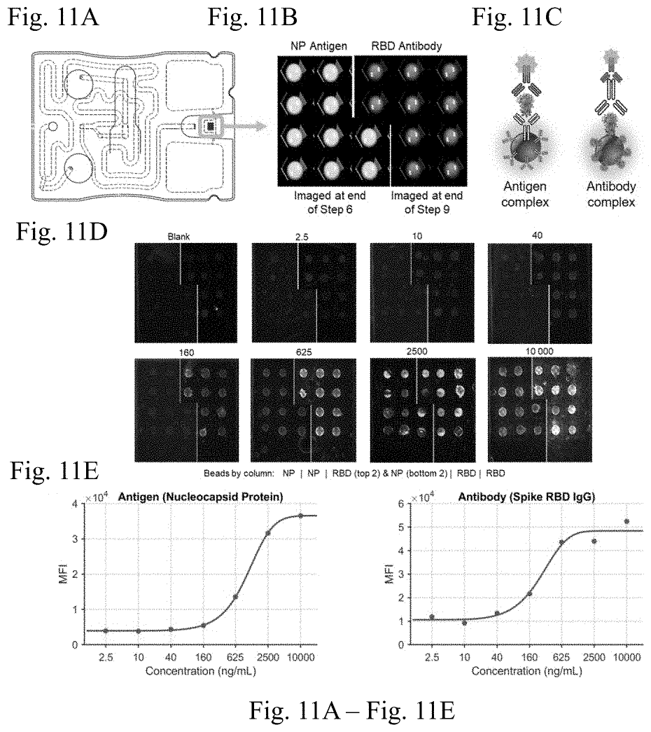

Patients scoring above the threshold on the pre-screening assessment will be recommended for an on-site POC combinatorial antigen/antibody test (FIG. 11). To demonstrate proof of concept, standard curves for antigen (SARS-CoV-2 nucleocapsid) and antibody (spike RBD) were completed with 4-fold serially diluted analyte spiked sample buffer, covering a range of high viral antigen and immune response load (10,000 ng/ml) to very low loads (2 ng/ml). Standard curves show a pattern of progressive fluorescence intensity and increasing signal-to-blank ratio (SBR), with intra-assay precision ranging from 7-25%. Initial LOD calculations suggest <100 ng for the antigen and antibody detection.

TABLE 5
Diagnostic performance of the full model (local positivity rate,
SpO2 ≤96%, temperature ≥99° F., race, and ethnicity)

| | Full Model | |
|---|---|---|
| AUC | 0.76 | (0.73-0.78) |
| Sensitivity | 0.90 | (0.89-0.91) |
| Specificity | 0.39 | (0.37-0.41) |
| PPV | 0.26 | (0.24-0.28) |
| NPV | 0.94 | (0.93-0.95) |

TABLE 6
Diagnostic performance of the preferred
model (case incidence rate)

| | Preferred Model | |
|---|---|---|
| AUC | 0.65 | (0.62-0.68) |
| Sensitivity | 0.90 | (0.88-0.91) |
| Specificity | 0.23 | (0.21-0.25) |
| PPV | 0.22 | (0.20-0.23) |
| NPV | 0.90 | (0.89-0.91) |

TABLE 7
Table of diagnostic performance for models discriminating COVID-19 positive
vs. negative (RT-PCR) in pre- and asymptomatic individuals. This table
corresponds to the data shown in FIG. 10 in the main text. Temperature
is body temperature ≥99° F. SpO2 is oxygen saturation ≤96%.
CIR is the case incidence rate. LPR is the local positivity rate.

| | AUC (95% CI) | |
|---|---|---|
| Temperature only | 0.52 | (0.49-0.55) |
| SpO2 only | 0.55 | (0.52-0.58) |
| CIR only (preferred model) | 0.65 | (0.62-0.68) |
| CIR + SpO2 | 0.67 | (0.64-0.70) |
| CIR + SpO2 + Temperature | 0.68 | (0.65-0.71) |
| CIR + SpO2 + Temperature + Race & Ethnicity | 0.71 | (0.68-0.74) |
| LPR only | 0.71 | (0.67-0.73) |
| LPR + SpO2 | 0.72 | (0.69-0.75) |
| LPR + SpO2 + Temperature | 0.72 | (0.70-0.75) |
| LPR + SpO2 + Temperature + Race & Ethnicity (full model) | 0.76 | (0.73-0.78) |

TABLE 8
Lasso logistic regression coefficients for the full model

| Predictor | β |
|---|---|
| (intercept) | −1.7505 |
| SpO2 ≤96% | 0.2822 |
| Temperature ≥99° F. | 0.1434 |
| Ethnicity - Hispanic | 0.6096 |
| Race - White | −0.4115 |
| Race - Asian | −0.5630 |
| Race - Black | −0.0160 |
| LPR | 0.7767 |

TABLE 9
Lasso logistic regression coefficients for the preferred model

| Predictor | β |
|---|---|
| (intercept) | −1.5113 |
| CIR | 0.5266 |

### Discussion

With dental health care delayed or interrupted, detrimental effects on oral as well as overall health may soon follow. Prolonged interruption in preventive care and treatment for early forms of dental disease may increase treatment complexity and cost. The screening approach described in this study can provide in near real time the COVID-19 status of each patient and employee at the dental office and, thus, significantly reduce the risk of spreading COVID-19.

Despite being the de facto method for COVID-19 screening in dental offices to date, temperature was found to be relatively ineffective at distinguishing which pre- or asymptomatic patients were infected. However, temperature checks may still play an important role in detecting symptomatic individuals who unknowingly visit the dental office with a fever. Likewise, measurements of SpO2 did not show significant improvements over temperature despite its potential importance in monitoring disease progression in confirmed COVID-19 cases.

One unexpected finding of this analysis was that when and where a person is being screened was the most important factor in predicting COVID-19 status. The local test positivity rate and case incidence rate were the strongest predictors of COVID-19 status, outperforming physiological and demographic factors. This result demonstrates the significance of time- and location-specific spread data within communities in estimating the pre-test probabilities for COVID-19 screening. This result may be especially relevant for large academic dental centers which see an influx of patients from a broader geographic region compared to community dental clinics.

Combining test positivity with race and ethnicity improved the performance (AUC 0.76); however, inclusion of racial and ethnic information are controversial in medical algorithms (Vyas, D A et al., 2020, N. Engl. J. Med. 383(9):874-882) and may not generalize well to less diverse populations. While other studies have found that COVID-19 disproportionately affects racial and ethnic minority groups, our study did not detect those differences as these data were largely represented by vulnerable communities served by NYU Langone's FHCs. In addition, while test positivity rate was a better predictor than incidence rate, the testing data available to date are only reliably available at the US state level, not the county level, and are, thus, inappropriate for risk assessment in states with an uneven geographical distribution of cases. For these reasons, we have designated the model with case incidence rate as the preferred model. One limitation of this current study is that these predictive models, while intended for dental screening, were trained using data from community health clinics and hospitals within the NYU FHC network. Near term future efforts are planned to externally validate the models for use in dental office settings.

The POC diagnostics are critical for successfully mitigating COVID-19 transmission risk in asymptomatic and/or presymptomatic populations. Expanding access to in situ testing capabilities adds significant convenience to the risk management infrastructure much needed in dental offices. While the current gold-standard RT-PCR detection techniques are highly valuable, the added time, cost, and demand-supply chain are bottlenecks for testing requirements. Convenient antigen testing combined with rapid antibody-based testing has much potential in covering these testing bottlenecks. In contrast to traditional lateral flow and ELISA techniques, the multiplexed microfluidics-based assay developed here has the potential to achieve high sensitivity in a convenient format with noninvasive sampling while maintaining high specificity. Any positive result on the antigen/antibody test may then be followed up with RT-PCR for confirmation.

Detecting SARS-CoV-2 from oro-/nasopharyngeal swabs requires high-quality specimens with sufficient amounts of intact viral RNA. However, viral loads in the respiratory tract have shown to be highly variable, leading to high false-negative rates. Recently, saliva has emerged as a promising alternative to nasopharyngeal swabs for COVID-19 diagnosis and monitoring (Kojima, N. et al., 2020, Clin. Infect. Dis., ciaa1589; Wyllie, A L et al., 2020, N. Engl. J. Med. 383(13):1283-1286) in which testing accuracy may be improved by saliva's more uniform availability of antigens and antibodies. The saliva sampling solution proposed here circumvents the aforementioned limitations of oro- and nasopharyngeal sampling as patients can self-collect saliva samples with minimal instruction at the POC.

A significant challenge with multiplexing is cross-reactivity between capture and detecting reagents, particularly in combining immunoassay formats. These issues can be mitigated through optimization of reagent sources, subtypes, blocking strategies, assay flow rates, and volumes. Further, limitations of this testing strategy include obtaining negative results in patients during their incubation period who later become infectious. Cost, complexity, and supply chain shortages are current bottlenecks for scaling SARS-CoV-2 testing. While this current work serves to demonstrate initial method validation and a promising implementation for high-risk settings requiring rapid, cost-effective, convenient, and accurate screening results, future work will involve further assessment of qualitative performance (sensitivity and specificity) and blinded validation of the combinatorial format with real patient samples confirmed by RT-PCR and lab-based serological testing methods.

To facilitate health policy decisions, governments across the globe use estimates of transmission rates, case numbers, and fatality rates. By conducting random antibody sampling of the general public, public health bodies could better estimate the true levels of exposure and resulting population immunity. For COVID-19, this would be a game changer, as true transmission and case fatality rates could be calculated to forecast the intensity and longevity of the pandemic to direct decision making. The highly accessible dental office would well serve the goal of identifying potential geographic regions of low population immunity to better allocate resources to prevent or manage transmission. These efforts are directed to COVID-19, but the same POC tools here described can be applied for other oral and systemic diseases. (McRae, M P et al., 2016, Acc. Chem. Res. 49(7):1359-1368), (McRae, M P et al., 2020, Cancer Cytopathology. 128(3):207-220)

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A device comprising one or more bioaffinity ligands specific for one or more biomarkers of a pathogen-mediated infection or disease or the disease severity of the pathogen-mediated infection or disease.

2. The device of claim 1, wherein the pathogen-mediated infection or disease is COVID-19.

3. The device of claim 1, wherein the device comprises an array of bead sensors, wherein each said bead sensor is a porous polymeric bead having an antibody or related bioaffinity ligand bound thereto.

4. The device of claim 2, wherein the biomarker of COVID-19 is selected from the group consisting of IgG, IgM, and SARS CoV-2 spike and wherein the biomarker of COVID-19 disease severity is selected from the group consisting of: CRP, PCT, CK-MB, c-TN-I, D-dimer, and NT-proBNP.

5. (canceled)

6. The device of claim 3, further comprising internal microfluidics on said substrate for carrying fluid to and from said bead sensors.

7. The device of claim 3, further comprising at least one reagent blister fluidly connected to said bead sensors.

8. The device of claim 3, further comprising positive and negative control bead sensors and calibrator bead sensors.

9. The device of claim 3, wherein every said bead sensor is present in said array in at least duplicate.

10. The device of claim 3, wherein said antibody or bioaffinity ligand is conjugated to said bead sensor via a linker.

11. The device of claim 3, further comprising:
a) one or more reagent chambers fluidly connected to and upstream of said array; and
b) one or more waste fluid chambers fluidly connected to and downstream of said array;
c) a sample inlet upstream and fluidly connected to said one or more reagent chambers; and
d) wherein each bead sensor is a porous polymeric bead of size between 50-300 μm±10%.

12. (canceled)

13. (canceled)

14. A method for diagnosing or treating a pathogen-mediated disease or infection, the method comprising obtaining a biological sample from a patient; and immunologically testing said sample to determine the of level of one or more biomarkers of the pathogen-mediated infection or one or more biomarkers of the disease severity of the pathogen-mediated infection.

15. The method of claim 14, wherein the pathogen-mediated infection or disease is COVID-19.

16. (canceled)

17. The method of claim 15, wherein the biomarker of COVID-19 is selected from the group consisting of IgG, IgM, and SARS CoV-2 spike, and wherein the biomarker of COVID-19 disease severity is selected from the group consisting of: CRP, PCT, CK-MB, c-TN-I, D-dimer, and NT-proBNP.

18. (canceled)

19. (canceled)

20. The method of claim 14, wherein the method further comprises performing an optimal clinical intervention, when the level of the one or more biomarkers are above a threshold level.

21. A method for screening a subject for the probability of SARS-CoV2 infection, comprising calculating a screening score for the subject, wherein the screening score is based upon a logistic regression model of one or more environmental, physiological, or demographic factors of the subject.

22. The method of claim 21, wherein the subject is a patient scheduled for a dental or medical procedure.

23. The method of claim 21, wherein the one or more environmental, physiological, or demographic factors of the subject comprises one or more of: body temperature, SpO2, race/ethnicity, local positivity rate of the subject's residence, case incidence rate of the subject's residence.

24. The method of claim 21, wherein the logistic regression model is a lasso logistic regression model.

25. The method of claim 21, further comprising obtaining a sample of the subject when the score surpasses a threshold; and assaying the sample for one or more antigens associated with SARS-CoV2 infection and one or more antibodies associated with SARS-CoV2 infection.

26. The method of claim 25, wherein assaying comprises contacting the sample to a point-of-care device that sequentially assays for the one or more antigens and the one or more antibodies.

27. (canceled)

28. (canceled)
